(19) United States
(12) Patent Application Publication
(10) Pub. No.: US 2025/0018056 A1
(43) Pub. Date: Jan. 16, 2025

(54) STRATEGIES FOR KNOCK-INS AT C3 SAFE HARBOR SITES

(71) Applicant: EmendoBio Inc., Wilmington, DE (US)

(72) Inventors: Rafi Emmanuel, Ramla (IL); Michal Golan Mashiach, Ness Ziona (IL); Joseph Dicken, Rehovot (IL)

(73) Assignee: EmendoBio Inc., Wilmington, DE (US)

(21) Appl. No.: 18/549,791

(22) PCT Filed: Mar. 10, 2022
(86) PCT No.: PCT/US2022/019701

§ 371 Date: Sep. 8, 2023

Related U.S. Application Data
(60) Provisional application No. 63/159,602, filed on Mar. 11, 2021.

(51) Int. Cl. A61K 48/00; A61P 1/16; C12N 9/22; C12N 15/11; C12N 15/90

(52) U.S. Cl. CPC A61K 48/005; A61P 1/16; C12N 9/22; C12N 15/111; C12N 15/902; C12N 2310/10

(57) ABSTRACT

RNA molecules comprising a guide sequence portion having 17-50 contiguous nucleotides in the sequence set forth in any one of SEQ ID NOs: 1-7,046 and compositions, methods, and uses thereof.

This application claims the benefit of U.S. Provisional Application No. 63/159,602, filed Mar. 11, 2021, the contents of which are hereby incorporated by reference.

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

### REFERENCE TO SEQUENCE LISTING

This application incorporates-by-reference nucleotide sequences which are present in the file named “220310_91702-A-PCT_Sequence_Listing_AWG.txt”, which is 1,526 kilobytes in size, and which was created on Feb. 28, 2022 in the IBM-PC machine format, having an operating system compatibility with MS-Windows, which is contained in the text file filed Mar. 10, 2022 as part of this application.

### BACKGROUND OF INVENTION

There are several classes of DNA variation in the human genome, including insertions and deletions, differences in the copy number of repeated sequences, and single nucleotide polymorphisms (SNPs). A SNP is a DNA sequence variation occurring when a single nucleotide (adenine (A), thymine (T), cytosine (C), or guanine (G)) in the genome differs between human subjects or paired chromosomes in an individual. Over the years, the different types of DNA variations have been the focus of the research community either as markers in studies to pinpoint traits or disease causation or as potential causes of genetic disorders.

DNA variations may also be utilized to target a specific DNA site in a cell. For example, an allele-specific Complement component 3 (C3) safe harbor site may be targeted to introduce a sequence to the site without causing deleterious disruptions. Such targeting strategies may be utilized to enable C3 promoter mediated expression of the introduced sequence in order to treat liver-related disorders.

### SUMMARY OF THE INVENTION

Disclosed herein are strategies to enable the expression of a gene, or portion thereof, under control of the C3 promoter optionally without knocking-out C3 gene expression.

The present disclosure may utilize at least one naturally occurring nucleotide difference or polymorphism (e.g., single nucleotide polymorphism (SNP)) for distinguishing/discriminating between two alleles of a C3 gene.

The present disclosure also provides a method for modifying in a cell an allele of the Complement component 3 (C3) gene, the method comprising
introducing to the cell a composition comprising:
   at least one CRISPR nuclease or a nucleotide sequence encoding a CRISPR nuclease; and
   a RNA molecule comprising a guide sequence portion having 17-50 nucleotides or a nucleotide sequence encoding the same,
wherein a complex of the CRISPR nuclease and the RNA molecule affects a double strand break in the allele of the C3 gene.

In some embodiments, the composition also comprises a donor molecule, wherein a sequence of nucleotides from the donor molecule is inserted or copied at or near the double strand break site. Similarly, in some embodiments, the composition further comprises a donor molecule containing a sequence of nucleotides that is introduced at the double strand break site such that the expression of the introduced sequence is mediated by the promoter of the C3 gene.

According to embodiments of the present invention, there is provided a first RNA molecule comprising a guide sequence portion having 17-50 contiguous nucleotides containing nucleotides in the sequence set forth in any one of SEQ ID NOs: 1-7,046. In some embodiments, the composition further comprises a CRISPR nuclease. In some embodiments, the composition further comprises a donor molecule.

According to embodiments of the present invention, there is provided a cell modified by any one of the methods described herein. In some embodiments, the cell is a stem cell. In some embodiments, the cell is a liver cell. In some embodiments, the cell is a hepatocyte. In some embodiments, the cell is an iPS-derived hepatocyte.

In some embodiments, the delivering of any one of the compositions described herein to the cell is performed in vitro, ex vivo, or in vivo. In some embodiments, the method is performed ex vivo and the cell is provided/explanted from an individual patient. In some embodiments, the method further comprises the step of introducing the resulting cell, with a modified or edited C3 allele, into the individual patient (e.g. autologous transplantation).

According to some embodiments of the present invention, there is provided a method for treating a liver-related disorder, the method comprising delivering to a cell of a subject having a liver-related disorder a composition comprising an RNA molecule comprising a guide sequence portion having 17-50 contiguous nucleotides containing nucleotides in the sequence set forth in any one of SEQ ID NOs: 1-7,046 and a CRISPR nuclease. In some embodiments, the composition further comprises a donor molecule.

According to some embodiments of the present invention, there is provided use of a composition comprising an RNA molecule comprising a guide sequence portion having 17-50 contiguous nucleotides containing nucleotides in the sequence set forth in any one of SEQ ID NOs: 1-7,046 and a CRISPR nuclease for modifying or editing a C3 allele in a cell, comprising delivering to the cell the composition comprising an RNA molecule comprising a guide sequence portion having 17-50 contiguous nucleotides containing nucleotides in the sequence set forth in any one of SEQ ID NOs: 1-7,046 and a CRISPR nuclease. In some embodiments, the composition further comprises a donor molecule.

According to embodiments of the present invention, there is provided a medicament comprising an RNA molecule comprising a guide sequence portion having 17-50 contiguous nucleotides containing nucleotides in the sequence set forth in any one of SEQ ID NOs: 1-7,046 and a CRISPR nuclease for use in modifying or editing a C3 allele in a cell, wherein the medicament is administered by delivering to the cell the composition comprising an RNA molecule comprising a guide sequence portion having 17-50 contiguous nucleotides containing nucleotides in the sequence set forth in any one of SEQ ID NOs: 1-7,046 and a CRISPR nuclease. In some embodiments, the medicament further comprises a donor molecule.

According to some embodiments of the present invention, there is provided use of a composition comprising an RNA molecule comprising a guide sequence portion having 17-50 contiguous nucleotides containing nucleotides in the sequence set forth in any one of SEQ ID NOs: 1-7,046 and a CRISPR nuclease for treating ameliorating or preventing a liver-related disorder, comprising delivering to a cell of a subject having or at risk of having a liver-related disorder the composition of comprising an RNA molecule comprising a guide sequence portion having 17-50 contiguous nucleotides containing nucleotides in the sequence set forth in any one of SEQ ID NOs: 1-7,046 and a CRISPR nuclease. In some embodiments, the composition further comprises a donor molecule. In some embodiments, the method is performed ex vivo and the cell is provided/explanted from the subject. In some embodiments, the method further comprises the step of introducing the resulting cell, with the modified or edited C3 allele, into the subject (e.g. autologous transplantation).

According to some embodiments of the present invention, there is provided a medicament comprising the composition comprising an RNA molecule comprising a guide sequence portion having 17-50 contiguous nucleotides containing nucleotides in the sequence set forth in any one of SEQ ID NOs: 1-7,046 and a CRISPR nuclease for use in treating ameliorating or preventing a liver-related disorder, wherein the medicament is administered by delivering to a cell of a subject having or at risk of having a liver-related disorder the composition comprising an RNA molecule comprising a guide sequence portion having 17-50 contiguous nucleotides containing nucleotides in the sequence set forth in any one of SEQ ID NOs: 1-7,046 and a CRISPR nuclease. In some embodiments, the medicament further comprises a donor molecule.

According to some embodiments of the present invention, there is provided a kit for modifying or editing C3 allele in a cell, comprising an RNA molecule comprising a guide sequence portion having 17-50 contiguous nucleotides containing nucleotides in the sequence set forth in any one of SEQ ID NOs: 1-7,046, a CRISPR nuclease, and/or a tracrRNA molecule; and instructions for delivering the RNA molecule; CRISPR nuclease, and/or the tracrRNA to the cell. In some embodiments, the kit further comprises a donor molecule.

According to some embodiments of the present invention, there is provided a kit for treating a liver-related disorder in a subject, comprising an RNA molecule comprising a guide sequence portion having 17-50 contiguous nucleotides containing nucleotides in the sequence set forth in any one of SEQ ID NOs: 1-7,046, a CRISPR nuclease, and/or a tracrRNA molecule; and instructions for delivering the RNA molecule; CRISPR nuclease, and/or the tracrRNA to a cell of a subject having or at risk of having a liver-related disorder. In some embodiments, the kit further comprises a donor molecule.

### BRIEF DESCRIPTION OF THE DRAWINGS

FIG. 1: Editing activity of RNA guide molecules targeting intron 1 of the C3 gene in HeLa cells. Cells were harvested 72 hours post-DNA transfection, genomic DNA was extracted, and the targeted region was amplified and analyzed for mutations by next-generation sequencing (NGS). The graph represents the % of editing ±standard deviation of three independent transfections.

FIG. 2: Activity of “g6” and “g11” guide molecules, which each intron 1 of the C3 gene. OMNI-79 guides were transfected in HeLa cells. RNPs with OMNI-50 protein and OMNI-50 synthetic RNA guide molecules were electroporated into HepG2 cells. Cells were harvested 72 hours post DNA transfection, genomic DNA was extracted, and the targeted region was amplified and analyzed for mutations by next-generation sequencing (NGS). The graph represents the % of editing ±standard deviation of three independent transfections or two independent RNP electroporations.

FIG. 3: Relative amount of C3 mRNA in edited samples compared to control samples. Cells were harvested seven (7) days after electroporation. RNA was then extracted and processed for RT-qPCR. The graph represents the relative amount of C3 mRNA in edited samples compared to control samples, ±standard deviation of two independent RNP electroporations.

FIG. 4: Insertion of GFP into a C3 endogenous locus by homology directed repair (HDR). Exon 1 encodes a signal peptide that is cleaved before secretion. GFP is spliced together with the exon 1 signal peptide that is to be secreted. In the schematic diagram, “Left HA” and “Right HA” refer to left and right homology arms, respectively, “SA” refers to a splice acceptor site, and “PolyA” refers to a polyadenylation signal.

FIG. 5: GFP expression 72 hours post-infection as measured by flow cytometry. HepG2 cells were infected with the indicated AAV viral particles, containing either a CRISPR nuclease (OMNI-79 V5570) and a guide (C3 g11) or a GFP homology directed repair (HDR) template. AAV containing a DNA molecule encoding GFP under a constitutive promoter was used as a positive control for infection efficiency evaluation. No GFP expression was detected in cells infected with the HDR template only, which indicates no off-target integration. Graphs represents the average ±standard deviation (n=3) of % GFP positive cells.

FIG. 6: GFP expression in HepG2 cells fifteen (15) days post-infection as measured by flow cytometry. Notably, the population of GFP positive cells increased in its frequency.

FIG. 7: Plate Reader Measurement of GFP Secretion in HepG2 Cells Fifteen (15) Days Post-Treatment. Fourteen (14) days after infection, cell media was replaced to Opti-MEM® to reduce autofluorescence and at Day 15 cell media was collected to measure GFP fluorescence. In all HDR positive conditions, GFP fluorescence was above the background of the non-treated (NT) cells.

FIG. 8: GFP integration following editing after electroporation as measured by flow cytometry detection of GFP expression eight (8) days post-treatment. HepG2 cells were electroporated with an RNP comprising a OMNI-50 nuclease and a guide (C3 g11), and infected with AAV containing a GFP HDR template two (2) hours after electroporation. AAV containing a DNA molecule encoding GFP under a constitutive promoter was used as a positive control for infection efficiency evaluation. GFP was detected in cells electroporated with RNP and infected with HDR template. The graph represents average ±standard deviation (n=2) of % GFP positive cells.

FIG. 9: Plate Reader Measurement of GFP Secretion in HepG2 Cells Eight (8) Days Post-Treatment. Seven (7) days after infection, cell media was replaced to Opti-MEM® to reduce autofluorescence and at Day 8 cell media was collected to measure GFP fluorescence. Non-specific fluorescence in a non-treated (NT) sample was subtracted from all samples.

### DETAILED DESCRIPTION

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

Unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

In the description and claims of the present application, each of the verbs, “comprise,” “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

The term “homology-directed repair” or “HDR” refers to a mechanism for repairing DNA damage in cells, for example, during repair of double-stranded and single-stranded breaks in DNA. HDR requires nucleotide sequence homology and uses a “nucleic acid template” (nucleic acid template or donor template used interchangeably herein) to repair the sequence where the double-stranded or single break occurred (e.g., DNA target sequence). This results in the transfer of genetic information from, for example, the nucleic acid template to the DNA target sequence. HDR may result in alteration of the DNA target sequence (e.g., insertion, deletion, mutation) if the nucleic acid template sequence differs from the DNA target sequence and part or all of the nucleic acid template polynucleotide or oligonucleotide is incorporated into the DNA target sequence. In some embodiments, an entire nucleic acid template polynucleotide, a portion of the nucleic acid template polynucleotide, or a copy of the nucleic acid template is integrated at the site of the DNA target sequence.

The terms “nucleic acid template” and “donor”, refer to a nucleotide sequence that is inserted or copied into a genome. The nucleic acid template comprises a nucleotide sequence, e.g., of one or more nucleotides, that will be added to or will template a change in the target nucleic acid or may be used to modify the target sequence. A nucleic acid template sequence may be of any length, for example between 2 and 10,000 nucleotides in length. A nucleic acid template may be a single stranded nucleic acid, a double stranded nucleic acid. In some embodiments, the nucleic acid template comprises a nucleotide sequence, e.g., of one or more nucleotides, that corresponds to wild type sequence of the target nucleic acid, e.g., of the target position. In some embodiments, the nucleic acid template comprises a nucleotide sequence, e.g., of one or more ribonucleotides, that corresponds to wild type sequence of the target nucleic acid, e.g., of the target position. In some embodiments, the nucleic acid template comprises modified nucleotides.

Insertion of an exogenous sequence (also called a “donor sequence,” donor template,” “donor molecule” or “donor”) can also be carried out. For example, a donor sequence can contain a non-homologous sequence flanked by two regions of homology to allow for efficient homology directed repair (HDR) at the location of interest. Additionally, donor sequences can comprise a vector molecule containing sequences that are not homologous to the region of interest in cellular chromatin. A donor molecule can contain several, discontinuous regions of homology to cellular chromatin. For example, for targeted insertion of sequences not normally present in a region of interest, said sequences can be present in a donor nucleic acid molecule and flanked by regions of homology to sequence in the region of interest. A donor molecule may be any length, for example ranging from several bases e.g. 10-20 bases to multiple kilobases in length.

The donor polynucleotide can be DNA or RNA, single-stranded and/or double-stranded and can be introduced into a cell in linear or circular form. See, e.g., U.S. Patent Publication Nos. 2010/0047805; 2011/0281361; 2011/0207221; and 2019/0330620. See also Anzalone et al. (2019). If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al. (1987) and Nehls et al. (1996). Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A donor sequence may be an oligonucleotide and be used for targeted alteration of an endogenous sequence. The oligonucleotide may be introduced to the cell on a vector, may be electroporated into the cell, or may be introduced via other methods known in the art. Donor polynucleotides can be introduced as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

As used herein, the term “modified cells” refers to cells in which a double strand break is affected by a complex of an RNA molecule and the CRISPR nuclease as a result of hybridization with the target sequence, i.e. on-target hybridization. The term “modified cells” may further encompass cells in which an edit or modification, including the introduction of an exogenous sequence, was affected following the double strand break.

This invention provides a modified cell or cells obtained by use of any of the methods described herein. In an embodiment these modified cell or cells are capable of giving rise to progeny cells. In an embodiment these modified cell or cells are capable of giving rise to progeny cells after engraftment. As a non-limiting example, the modified cells may be hematopoietic stem cells (HSCs), or any cell suitable for an allogenic cell transplant or autologous cell transplant. As a non-limiting example, the modified cells may be stem cells, liver cells, hepatocytes, or iPS-derived hepatocytes.

This invention also provides a composition comprising these modified cells and a pharmaceutically acceptable carrier. Also provided is an in vitro or ex vivo method of preparing this, comprising mixing the cells with the pharmaceutically acceptable carrier.

As used herein, the term “targeting sequence” or “targeting molecule” refers a nucleotide sequence or molecule comprising a nucleotide sequence that is capable of hybridizing to a specific target sequence, e.g., the targeting sequence has a nucleotide sequence which is at least partially complementary to the sequence being targeted along the length of the targeting sequence. The targeting sequence or targeting molecule may be part of an RNA molecule that can form a complex with a CRISPR nuclease, either alone or in combination with other RNA molecules, with the targeting sequence serving as the targeting portion of the CRISPR complex. When the molecule having the targeting sequence is present contemporaneously with the CRISPR molecule, the RNA molecule, alone or in combination with an additional one or more RNA molecules (e.g. a tracrRNA molecule), is capable of targeting the CRISPR nuclease to the specific target sequence. As non-limiting example, a guide sequence portion of a CRISPR RNA molecule or single-guide RNA molecule may serve as a targeting molecule. Each possibility represents a separate embodiment. A targeting sequence can be custom designed to target any desired sequence.

The term “targets” as used herein, refers to preferentially hybridizing a targeting sequence of a targeting molecule to a nucleic acid having a targeted nucleotide sequence. It is understood that the term “targets” encompasses variable hybridization efficiencies, such that there is preferential targeting of the nucleic acid having the targeted nucleotide sequence, but unintentional off-target hybridization in addition to on-target hybridization might also occur. It is understood that where an RNA molecule targets a sequence, a complex of the RNA molecule and a CRISPR nuclease molecule targets the sequence for nuclease activity.

The “guide sequence portion” of an RNA molecule refers to a nucleotide sequence that is capable of hybridizing to a specific target DNA sequence, e.g., the guide sequence portion has a nucleotide sequence which is partially or fully complementary to the DNA sequence being targeted along the length of the guide sequence portion. In some embodiments, the guide sequence portion is 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length, or approximately 17-50, 17-49, 17-48, 17-47, 17-46, 17-45, 17-44, 17-43, 17-42, 17-41, 17-40, 17-39, 17-38, 17-37, 17-36, 17-35, 17-34, 17-33, 17-31, 17-30, 17-29, 17-28, 17-27, 17-26, 17-25, 17-24, 17-22, 17-21, 18-25, 18-24, 18-23, 18-22, 18-21, 19-25, 19-24, 19-23, 19-22, 19-21, 19-20, 20-22, 18-20, 20-21, 21-22, or 17-20 nucleotides in length. Preferably, the entire length of the guide sequence portion is fully complementary to the DNA sequence being targeted along the length of the guide sequence portion. The guide sequence portion may be part of an RNA molecule that can form a complex with a CRISPR nuclease with the guide sequence portion serving as the DNA targeting portion of the CRISPR complex. When the RNA molecule having the guide sequence portion is present contemporaneously with the CRISPR molecule, alone or in combination with an additional one or more RNA molecules (e.g. a tracrRNA molecule), the RNA molecule is capable of targeting the CRISPR nuclease to the specific target DNA sequence. Accordingly, a CRISPR complex can be formed by direct binding of the RNA molecule having the guide sequence portion to a CRISPR nuclease or by binding of the RNA molecule having the guide sequence portion and an additional one or more RNA molecules to the CRISPR nuclease. Each possibility represents a separate embodiment. A guide sequence portion can be custom designed to target any desired sequence. Accordingly, a molecule comprising a “guide sequence portion” is a type of targeting molecule. In some embodiments, the guide sequence portion comprises a sequence that is the same as, or differs by no more than 1, 2, 3, 4, or 5 nucleotides from, a guide sequence portion described herein, e.g., a guide sequence set forth in any of SEQ ID NOs:1-7,046. Each possibility represents a separate embodiment. In some of these embodiments, the guide sequence portion comprises a sequence that is the same as a sequence set forth in any of SEQ ID NOs:1-7,046. Throughout this application, the terms “guide molecule,” “RNA guide molecule,” “guide RNA molecule,” and “gRNA molecule” are synonymous with a molecule comprising a guide sequence portion.

The term “non-discriminatory” as used herein refers to a guide sequence portion of an RNA molecule that targets a specific DNA sequence that is common in both alleles of a gene. For example, a non-discriminatory guide sequence portion is capable of targeting both alleles of a gene present in a cell.

In embodiments of the present invention, an RNA molecule comprises a guide sequence portion having 17-50 contiguous nucleotides containing nucleotides in the sequence set forth in any one of SEQ ID NOs: 1-7,046.

The RNA molecule and or the guide sequence portion of the RNA molecule may contain modified nucleotides. Exemplary modifications to nucleotides or polynucleotides may be synthetic and encompass polynucleotides which contain nucleotides comprising bases other than the naturally occurring adenine, cytosine, thymine, uracil, or guanine bases. Modifications to polynucleotides include polynucleotides which contain synthetic, non-naturally occurring nucleosides e.g., locked nucleic acids. Modifications to polynucleotides may be utilized to increase or decrease stability of an RNA. An example of a modified polynucleotide is an mRNA containing 1-methyl pseudo-uridine. For examples of modified polynucleotides and their uses, see U.S. Pat. No. 8,278,036, PCT International Publication No. WO/2015/006747, and Weissman and Kariko (2015), each of which is hereby incorporated by reference.

As used herein, “contiguous nucleotides” set forth in a SEQ ID NO refers to nucleotides in a sequence of nucleotides in the order set forth in the SEQ ID NO without any intervening nucleotides.

In embodiments of the present invention, the guide sequence portion may be 25 nucleotides in length and contain 20-22 contiguous nucleotides in the sequence set forth in any one of SEQ ID NOs: 1-7,046. In embodiments of the present invention, the guide sequence portion may be less than 22 nucleotides in length. For example, in embodiments of the present invention the guide sequence portion may be 17, 18, 19, 20, or 21 nucleotides in length. In such embodiments the guide sequence portion may consist of 17, 18, 19, 20, or 21 nucleotides, respectively, in the sequence of 17-22 contiguous nucleotides set forth in any one of SEQ ID NOs: 1-7,046. For example, a guide sequence portion having 17 nucleotides in the sequence of 17 contiguous nucleotides set forth in SEQ ID NO: 7,047 may consist of any one of the following nucleotide sequences (nucleotides excluded from the contiguous sequence are marked in strike-through):

(SEQ ID NO: 7,047)
AAAAAAAUGUACUUGGUUCC
17 nucleotide guide sequence 1:
(SEQ ID NO: 7,048) [symbol]
AAAAUGUACUUGGUUCC
17 nucleotide guide sequence 2:
(SEQ ID NO: 7,049) [symbol]
AAAAAUGUACUUGGUUC [symbol]
17 nucleotide guide sequence 3:
(SEQ ID NO: 7,050) [symbol]
AAAAAAUGUACUUGGUU [symbol]
17 nucleotide guide sequence 4:
(SEQ ID NO: 7,051)
AAAAAAAUGUACUUGGU

[symbol]

In embodiments of the present invention, the guide sequence portion may be greater than 20 nucleotides in length. For example, in embodiments of the present invention the guide sequence portion may be 21, 22, 23, 24, 25, 26, 27, 28, 29, or 30 nucleotides in length. In such embodiments the guide sequence portion comprises 17-50 nucleotides containing the sequence of 20, 21 or 22 contiguous nucleotides set forth in any one of SEQ ID NOs: 1-7,046 and additional nucleotides fully complimentary to a nucleotide or sequence of nucleotides adjacent to the 3′ end of the target sequence, 5′ end of the target sequence, or both.

In embodiments of the present invention a CRISPR nuclease and an RNA molecule comprising a guide sequence portion form a CRISPR complex that binds to a target DNA sequence to effect cleavage of the target DNA sequence. CRISPR nucleases, e.g. Cpf1, may form a CRISPR complex comprising a CRISPR nuclease and RNA molecule without a further tracrRNA molecule. Alternatively, CRISPR nucleases, e.g. Cas9, may form a CRISPR complex between the CRISPR nuclease, an RNA molecule, and a tracrRNA molecule. A guide sequence portion, which comprises a nucleotide sequence that is capable of hybridizing to a specific target DNA sequence, and a sequence portion that participates in CRIPSR nuclease binding, e.g. a tracrRNA sequence portion, can be located on the same RNA molecule. Alternatively, a guide sequence portion may be located on one RNA molecule and a sequence portion that participates in CRIPSR nuclease binding, e.g. a tracrRNA portion, may located on a separate RNA molecule. A single RNA molecule comprising a guide sequence portion (e.g. a DNA-targeting RNA sequence) and at least one CRISPR protein-binding RNA sequence portion (e.g. a tracrRNA sequence portion), can form a complex with a CRISPR nuclease and serve as the DNA-targeting molecule. In some embodiments, a first RNA molecule comprising a DNA-targeting RNA portion, which includes a guide sequence portion, and a second RNA molecule comprising a CRISPR protein-binding RNA sequence interact by base pairing to form an RNA complex that targets the CRISPR nuclease to a DNA target site or, alternatively, are fused together to form an RNA molecule that complexes with the CRISPR nuclease and targets the CRISPR nuclease to a DNA target site.

In embodiments of the present invention, an RNA molecule comprising a guide sequence portion may further comprise the sequence of a tracrRNA molecule. Such embodiments may be designed as a synthetic fusion of the guide portion of the RNA molecule and the trans-activating crRNA (tracrRNA). (See Jinek et al., 2012). In such an embodiment, the RNA molecule is a single-guide RNA (sgRNA) molecule. Embodiments of the present invention may also form CRISPR complexes utilizing a separate tracrRNA molecule and a separate RNA molecule comprising a guide sequence portion. In such embodiments the tracrRNA molecule may hybridize with the RNA molecule via basepairing and may be advantageous in certain applications of the invention described herein.

The term “tracr mate sequence” refers to a sequence sufficiently complementary to a tracrRNA molecule so as to hybridize to the tracrRNA via basepairing and promote the formation of a CRISPR complex. (See U.S. Pat. No. 8,906,616). In embodiments of the present invention, the RNA molecule may further comprise a portion having a tracr mate sequence.

A “gene,” for the purposes of the present disclosure, includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Eukaryotic” cells include, but are not limited to, fungal cells (such as yeast), plant cells, animal cells, mammalian cells and human cells.

The term “nuclease” as used herein refers to an enzyme capable of cleaving the phosphodiester bonds between the nucleotide subunits of nucleic acid. A nuclease may be isolated or derived from a natural source. The natural source may be any living organism. Alternatively, a nuclease may be a modified or a synthetic protein which retains the phosphodiester bond cleaving activity. Gene modification can be achieved using a nuclease, for example a CRISPR nuclease.

According to embodiments of the present invention, there is provided an RNA molecule comprising a guide sequence portion (e.g. a targeting sequence) comprising a nucleotide sequence that is fully or partially complementary to a target sequence comprising a SNP position (REF/SNP sequence) located in or near an allele of the C3 gene. In some embodiments, the guide sequence portion of the RNA molecule consists of 16, 17, 18, 19, 20, 21, 22, 23, 24, 25, 26, or more than 26 nucleotides. In some embodiments the guide sequence portion is configured to target a CRISPR nuclease to a target sequence and provide a cleavage event, by a CRISPR nuclease complexed therewith, selected from a double-strand break and a single-strand break within 500, 400, 300, 200, 100, 50, 25, or 10 nucleotides of a C3 target site. In some embodiments, the RNA molecule is a guide RNA molecule such as a crRNA molecule or a single-guide RNA molecule

As used herein, the term “HSC” refers to both hematopoietic stem cells and hematopoietic stem progenitor cells. Non-limiting examples of stem cells include bone marrow cells, myeloid progenitor cells, a multipotent progenitor cells, and lineage restricted progenitor cells.

As used herein, “progenitor cell” refers to a lineage cell that is derived from stem cell and retains mitotic capacity and multipotency (e.g., can differentiate or develop into more than one but not all types of mature lineage of cell). As used herein “hematopoiesis” or “hemopoiesis” refers to the formation and development of various types of blood cells (e.g., red blood cells, megakaryocytes, myeloid cells (e.g., monocytes, macrophages and neutrophil), and lymphocytes) and other formed elements in the body (e.g., in the bone marrow).

The term “single nucleotide polymorphism (SNP) position”, as used herein, refers to a position in which a single nucleotide DNA sequence variation occurs between members of a species, or between paired chromosomes in an individual. In the case that a SNP position exists at paired chromosomes in an individual, a SNP on one of the chromosomes is a “heterozygous SNP.” The term SNP position refers to the particular nucleic acid position where a specific variation occurs and encompasses both a sequence including the variation from the most frequently occurring base at the particular nucleic acid position (also referred to as “SNP” or alternative “ALT”) and a sequence including the most frequently occurring base at the particular nucleic acid position (also referred to as reference, or “REF”). Accordingly, the sequence of a SNP position may reflect a SNP (i.e. an alternative sequence variant relative to a consensus reference sequence within a population), or the reference sequence itself.

According to some embodiments of the present invention, there is provided a method for modifying in a cell an allele of the Complement component 3 (C3) gene, the method comprising
introducing to the cell a composition comprising:
   at least one CRISPR nuclease or a nucleotide sequence encoding a CRISPR nuclease; and
   a RNA molecule comprising a guide sequence portion having 17-50 nucleotides or a nucleotide sequence encoding the same,
wherein a complex of the CRISPR nuclease and the RNA molecule affects a double strand break in the allele of the C3 gene.

In some embodiments, the composition also comprises a donor molecule. In some embodiments, a sequence of nucleotides from the donor molecule is inserted or copied at or near the double strand break site.

In some embodiments, the composition further comprises a donor molecule containing a sequence of nucleotides that is introduced at the double strand break site such that the expression of the introduced sequence is mediated by the promoter of the C3 gene.

In some embodiments, the introduced sequence is a sequence from a ATP7B, A1AT, G6PC, SERPINA, LDLR, TTR, omithine transcarbamylase, argininosuccinic acid synthetase, arginase, argininosuccinase, carbamoyl phosphate synthetase, and N-acetylglutamate synthetase gene.

In some embodiments, the RNA molecule targets the CRISPR nuclease to a SNP position of a C3 allele.

In some embodiments, the SNP position is any one of rs17030, rs199713383, and rs35473940.

In some embodiments, the guide sequence portion of the first RNA molecule comprises 17-50 contiguous nucleotides containing nucleotides in the sequence set forth in any one of SEQ ID NOs: 1-7,046 that targets a SNP position of the C3 allele.

In some embodiments, the SNP position contains a heterozygous SNP.

In some embodiments, the SNP position is in an exon or intron of the C3 allele.

In some embodiments, the RNA molecule comprises a non-discriminatory guide portion that targets both C3 alleles.

In some embodiments, the RNA molecule comprises a non-discriminatory guide portion that targets any one of a non-discriminatory guide portion that targets Intron 1 of C3 or a 3′ untranslated region (3′ UTR) of C3.

In some embodiments, the RNA molecule comprises a non-discriminatory guide portion that targets a sequence that is located within a genomic range selected from any one of 19:6677732-6677881 and 19:6719404-6720515.

the guide sequence portion of the RNA molecule comprises 17-50 contiguous nucleotides containing nucleotides in the sequence set forth in any one of SEQ ID NOs: 1-7,046.

In some embodiments, the guide sequence portion of the RNA molecule comprises 1, 2, 3, 4, or 5 nucleotide mismatches relative to a fully-complementary C3 target sequence.

In some embodiments, the guide sequence portion of the RNA molecule comprises 17-50 contiguous nucleotides containing nucleotides in the sequence set forth in any one of SEQ ID NOs: 1-7,046 modified to contain 1, 2, 3, 4, or 5 nucleotide mismatches relative to a fully-complementary C3 target sequence.

In some embodiments, the guide sequence portion provides higher targeting specificity to the complex of the CRISPR nuclease and the RNA molecule relative to a guide sequence portion that has higher complementarity to an allele of the C3 gene.

According to embodiments of the present invention, there is provided a modified cell obtained by the method of any one of the embodiments presented herein.

According to some embodiments, the cell is a stem cell, liver cell, hepatocyte, or iPS-derived hepatocyte.

According to embodiments of the present invention, there is provided a RNA molecule comprising a guide sequence portion having 17-50 contiguous nucleotides containing nucleotides in the sequence set forth in any one of SEQ ID NOs: 1-7,046.

According to embodiments of the present invention, there is provided a composition comprising the RNA molecule and at least one CRISPR nuclease.

In some embodiments, the RNA molecule comprises a guide sequence portion having 17-50 contiguous nucleotides containing nucleotides in the sequence set forth in any one of SEQ ID NOs: 1-7,046, or SEQ ID NOs: 1-7,046 modified to contain 1, 2, 3, 4, or 5 nucleotide mismatches relative to a fully-complementary C3 target sequence.

In some embodiments, the composition further comprises a donor molecule.

In some embodiments, the donor molecule contains a sequence from a ATP7B, A1AT, G6PC, SERPINA, LDLR, TTR, ornithine transcarbamylase, argininosuccinic acid synthetase, arginase, argininosuccinase, carbamoyl phosphate synthetase, and N-acetylglutamate synthetase gene.

According to embodiments of the present invention, there is provided a method for modifying or editing a C3 allele in a cell, the method comprising delivering to the cell the composition of any one of the embodiments presented herein.

According to embodiments of the present invention, there is provided a method for treating a liver-related disorder, the method comprising delivering to a cell of a subject having a liver-related disorder the composition or delivering to the subject modified cells of any one of the embodiments presented herein.

According to embodiments of the present invention, there is provided use of any one of the compositions presented herein for modifying or editing a C3 allele in a cell, comprising delivering to the cell the composition of any one of the embodiments presented herein.

According to embodiments of the present invention, there is provided a medicament comprising the composition of any one of the embodiments presented herein for use in modifying or editing a C3 allele in a cell, wherein the medicament is administered by delivering to the cell the composition of any one of the embodiments presented herein.

According to embodiments of the present invention, there is provided use of the composition or modified cells of any one of the embodiments presented herein for treating ameliorating or preventing a liver-related disorder, comprising delivering to a cell of a subject having or at risk of having a liver-related disorder the composition of any one of the embodiments presented herein or delivering to the subject a cell modified by any one of the methods presented herein.

According to embodiments of the present invention, there is provided a medicament comprising the composition of any one of the embodiments presented herein for use in treating ameliorating or preventing a liver-related disorder, wherein the medicament is administered by delivering to a cell of a subject having or at risk of having a liver-related disorder the composition of any one of the embodiments presented herein.

According to embodiments of the present invention, there is provided a kit for modifying or editing a C3 allele in a cell, comprising an RNA molecule of any one of the embodiments presented herein, a CRISPR nuclease, and/or a tracrRNA molecule; and instructions for delivering the RNA molecule; CRISPR nuclease, and/or the tracrRNA to the cell. In some embodiments, the kit further comprises a donor molecule and instructions for delivering the donor molecule to a cell.

According to embodiments of the present invention, there is provided a kit for treating a liver-related disorder in a subject, comprising an RNA molecule of any one of the embodiments presented herein, a CRISPR nuclease, and/or a tracrRNA molecule; and instructions for delivering the RNA molecule; CRISPR nuclease, and/or the tracrRNA to a cell of a subject having or at risk of having a liver-related disorder. In some embodiments, the kit further comprises a donor molecule and instructions for delivering the donor molecule to a cell.

According to embodiments of the present invention, there is provided a gene editing composition comprising an RNA molecule comprising a guide sequence portion having 17-50 contiguous nucleotides containing nucleotides in the sequence set forth in any one of SEQ ID NOs: 1-7,046. In some embodiments, the RNA molecule further comprises a portion having a sequence which binds to a CRISPR nuclease. In some embodiments, the sequence which binds to a CRISPR nuclease is a tracrRNA sequence.

In some embodiments, the RNA molecule further comprises a portion having a tracr mate sequence.

In some embodiments, the RNA molecule may further comprise one or more linker portions.

According to embodiments of the present invention, an RNA molecule may be up to 1000, 900, 800, 700, 600, 500, 450, 400, 350, 300, 290, 280, 270, 260, 250, 240, 230, 220, 210, 200, 190, 180, 170, 160, 150, 140, 130, 120, 110, or 100 nucleotides in length. Each possibility represents a separate embodiment. In embodiments of the present invention, the RNA molecule may be 17 up to 300 nucleotides in length, 100 up to 300 nucleotides in length, 150 up to 300 nucleotides in length, 100 up to 500 nucleotides in length, 100 up to 400 nucleotides in length, 200 up to 300 nucleotides in length, 100 to 200 nucleotides in length, or 150 up to 250 nucleotides in length. Each possibility represents a separate embodiment.

According to some embodiments of the present invention, the composition further comprises a tracrRNA molecule.

According to some embodiments of the present invention, there is provided a method for modifying or editing a C3 allele in a cell, the method comprising delivering to the cell a composition comprising an RNA molecule comprising a guide sequence portion having 17-50 contiguous nucleotides containing nucleotides in the sequence set forth in any one of SEQ ID NOs: 1-7,046 and a CRISPR nuclease. In some embodiments, the composition further comprises a donor molecule.

According to some embodiments of the present invention, there is provided a method for treating a disorder, the method comprising delivering to a cell of a subject having the disorder a composition comprising an RNA molecule comprising a guide sequence portion having 17-50 contiguous nucleotides containing nucleotides in the sequence set forth in any one of SEQ ID NOs: 1-7,046 and a CRISPR nuclease. In some embodiments, the composition further comprises a donor molecule. In some embodiments, the disorder is a liver-related disorder. In some embodiments, the disorder is a lysosomal storage disorder.

According to some embodiments of the present invention, there is provided a method for modifying a DNA target site in a liver cell of a subject, wherein the modification of the DNA target site induces the liver cell to secrete a desired protein encoded by the modification, the method comprising delivering to the cell of the subject a composition comprising an RNA molecule comprising a guide sequence portion having 17-50 contiguous nucleotides containing nucleotides in the sequence set forth in any one of SEQ ID NOs: 1-7,046 and a CRISPR nuclease. In some embodiments, the composition further comprises a donor molecule.

According to embodiments of the present invention, at least one CRISPR nuclease and the RNA molecule or RNA molecules are delivered to the subject and/or cells substantially at the same time or at different times.

In some embodiments, a tracrRNA molecule is delivered to the subject and/or cells substantially at the same time or at different times as the CRISPR nuclease and RNA molecule or RNA molecules.

According to embodiments of the present invention, the RNA molecule targets a SNP in an exon, a SNP in an intron, a SNP in the first intron that follows the first coding exon of the C3 gene, a sequence common to both alleles in an intron, a sequence common to both alleles in the first intron that follows the first coding exon of the C3 gene, a SNP in the promoter region, the start codon, the stop codon, or an untranslated region (UTR), or a sequence present in both C3 alleles.

According to embodiments of the present invention, the RNA molecule targets an alternative splicing signal sequence between an exon and an intron of a C3 allele.

According to embodiments of the present invention, the RNA molecule is non-discriminatory and targets a sequence present in both C3 alleles. In some embodiments, the sequence is present in both C3 alleles. In some embodiments, the sequence is present in an intron of the C3 gene. In some embodiments, the intron is the first intron that follows the first coding exon of the C3 gene.

The compositions and methods of the present disclosure may be utilized for treating, preventing, ameliorating, or slowing progression of a disorder, such as a genetic disorder. For example, non-limiting examples of such a disorder is a liver-related disorder or a lysosomal storage disorder.

Any one of, or combination of, the above-mentioned strategies for modifying or editing a C3 allele may be used in the context of the invention.

According to some embodiments, the present disclosure provides an RNA sequence (also referred to as an ‘RNA molecule’) which binds to or associates with and/or directs an RNA-guided DNA nuclease e.g., a CRISPR nuclease, to a target sequence comprising at least one nucleotide which differs between alleles (e.g., SNP) of a gene of interest (e.g., a sequence of one C3 allele which is not present in the other C3 allele).

In some embodiments, the method comprises contacting at least one allele of a gene of interest with a non-discriminatory RNA molecule, e.g. an RNA molecule comprising a guide sequence portion which is capable of targeting both alleles of a gene, and a CRISPR nuclease e.g., a Cas9 protein, wherein the non-discriminatory RNA molecule and the CRISPR nuclease associate with a nucleotide sequence of the at least one allele of the gene of interest, thereby modifying or editing the at least one allele. Notably, although biallelic cleavage may occur upon introduction of a non-discriminatory RNA molecule to a cell, insertion of a nucleotide sequence at a cleavage site may occur in only a single allele and not in both alleles. Accordingly, inducing biallelic cleavage with a non-discriminatory RNA molecule that targets an intron of the C3 gene may result in preservation of expression of an endogenous C3 gene from one allele and introduction of a nucleotide sequence, e.g. a nucleotide sequence from a donor molecule, in the other allele. Introduction of the nucleotide sequence in a C3 allele may or may not disrupt expression of the C3 encoded gene product at the C3 allele.

In some embodiments, the method comprises contacting an allele of a gene of interest with an allele-specific RNA molecule and a CRISPR nuclease e.g., a Cas9 protein, wherein the allele-specific RNA molecule and the CRISPR nuclease associate with a nucleotide sequence of the allele of the gene of interest which differs by at least one nucleotide from a nucleotide sequence of a different allele of the gene of interest, thereby modifying or editing the targeted allele.

In some embodiments, the allele-specific or non-discriminatory RNA molecule and a CRISPR nuclease is introduced to a cell encoding the gene of interest. In some embodiments, the cell encoding the gene of interest is in a mammalian subject.

In some embodiments, the RNA molecule targets a SNP which is highly prevalent in the population and exists in at least one C3 allele of an individual subject to be treated.

In some embodiments, the SNP is within an exon of the gene of interest. In such embodiments, a guide sequence portion of an RNA molecule is designed to associate with a sequence of an exon of the gene of interest.

In some embodiments, SNP is within an intron or the exon of the gene of interest. In some embodiments, the SNP is in close proximity to the splice site between an intron and an exon. In some embodiments, the close proximity to a splice site is 1, 2, 3, 4, 5, 6, 7, 8, 9, 10, 11, 12, 13, 14, 15, 16, 17, 18, 19, or 20 nucleotides upstream or downstream to the splice site. Each possibility represents a separate embodiment of the present invention. In such embodiments, a guide sequence portion of an RNA molecule may be designed to associate with a sequence of the gene of interest which comprises the splice site.

In some embodiments, methods of the disclosed invention are utilized for treating a subject having a disorder or disease. In such embodiments, the method results in improvement, amelioration, or prevention of the disease phenotype. In some embodiments, the disease or disorder is a liver-related disorder. In some embodiments, the disease or disorder is a lysosomal storage disorder.

Embodiments of compositions described herein include at least one CRISPR nuclease, RNA molecule(s) comprising a guide sequence portion, and a tracrRNA molecule, which may be separate or attached to an RNA molecule comprising a guide sequence portion, being effective in a subject or cells at the same time. The at least one CRISPR nuclease, RNA molecule(s) comprising a guide sequence portion, and tracrRNA may be delivered substantially at the same time or can be delivered at different times but have effect at the same time. For example, this includes delivering the CRISPR nuclease to the subject or cells before the RNA molecule comprising a guide sequence portion and/or tracrRNA is substantially extant in the subject or cells.

In some embodiments, the cell is a stem cell. In some embodiments, the cell is a liver cell. In some embodiments, the cell is a hepatocyte. In some embodiments, the cell is an iPS-derived hepatocyte.

### Genetic C3 Safe Harbor Knock-Ins to Treat Liver-Related Disorders

In some embodiments, methods of the present invention may be used to knock-in a sequence at a C3 safe harbor site, with C3-mediated expression of the knocked-in sequence being involved in or associated with treatment of a disorder or a disease.

As a non-limiting example, expression of the knocked-in sequence may be involved in or associated with treatment of a liver-related disorder. For example, the knocked in sequence may be a ATP7B, A1AT, G6PC, SERPINA, LDLR, TTR, ornithine transcarbamylase, argininosuccinic acid synthetase, arginase, argininosuccinase, carbamoyl phosphate synthetase, or N-acetylglutamate synthetase sequence, or a portion thereof.

In another example, a C3 DNA target site in a liver cell is modified such that the liver cell expresses and secretes a protein product encoded by the modification. These modified liver cells may be utilized, for example, to treat lysosomal storage diseases or other disorders. Accordingly, these modified cells serve as an alternative to traditional enzyme replacement therapies. As a non-limiting example, a liver cell may be modified to express Alpha Galactosidase A, Coagulation Factor IX, Coagulation Factor VII, Lysosomal Alpha Glucosidase, Fibrinogen, Phenylalanine 4 Hydroxylase, Alkaline Phosphatase, Glucosylceramidase, Beta Galactosidase, Porphobilinogen Deaminase, Arylsulfatase B, Beta Glucuronidase, Alpha N Acetylglucosaminidase, Lysosomal Alpha, Alpha L-Iduronidase, Mannosidase, Phosphatidylcholine Sterol Acyltransferase, N-Sulphoglucosamine Sulphohydrolase, Coagulation Factor X, N-Acetylgalactosamine-6-Sulfatase, Sphingomyelin Phosphodiesterase, A1AT, Iduronate-2-Sulfatase, Lysosomal Alpha Glucosidase, Cyclin Dependent Kinase Like 5, Prolow Density Lipoprotein Receptor Related Protein 1, Phenylalanine Ammonia Lyase, Protein Glutamine Gamma Glutamyltransferase K, Lysosomal Protective Protein, or a portion thereof.

C3 editing strategies include strategies that enable expression of a desired sequence under the control of a C3 promoter, optionally without knocking out the edited C3 alleles. This can be achieved by the following strategies: (1) knock-in in the first intron of a C3 allele, or the first intron that follows the first coding exon of the C3 allele; (2) allele-specific knock-in in the first intron of a C3 allele, or the first intron that follows the first coding exon of the C3 allele; (3) knock-in by replacement of the stop codon of a C3 allele; or (4) knock-in by replacing the stop codon of a C3 allele in an allele-specific manner.

### CRISPR Nucleases and PAM Recognition

In some embodiments, the sequence specific nuclease is selected from CRISPR nucleases, or a functional variant thereof. In some embodiments, the sequence specific nuclease is an RNA-guided DNA nuclease. In such embodiments, the RNA sequence which guides the RNA guided DNA nuclease (e.g., Cpf1) binds to and/or directs the RNA guided DNA nuclease to a target sequence comprising at least one nucleotide which differs between alleles of a C3 gene (e.g., SNP). In some embodiments, the CRISPR complex does not further comprise a tracrRNA. In a non-limiting example, in which the RNA guided DNA nuclease is a CRISPR protein, the at least one nucleotide which differs between C3 alleles may be within the PAM site and/or proximal to the PAM site within the region that the RNA molecule is designed to hybridize to. A skilled artisan will appreciate that RNA molecules can be engineered to bind to a target of choice in a genome by commonly known methods in the art.

The term “PAM” as used herein refers to a nucleotide sequence of a target DNA located in proximity to the targeted DNA sequence and recognized by the CRISPR nuclease complex. The PAM sequence may differ depending on the nuclease identity. In addition, there are CRISPR nucleases that can target almost all PAMs. In some embodiments of the present invention, a CRISPR system utilizes one or more RNA molecules having a guide sequence portion to direct a CRISPR nuclease to a target DNA site via Watson-Crick base-pairing between the guide sequence portion and the protospacer on the target DNA site, which is next to the protospacer adjacent motif (PAM), which is an additional requirement for target recognition. The CRISPR nuclease then mediates cleavage of the target DNA site to create a double-stranded break within the protospacer. In a non-limiting example, a type II CRISPR system utilizes a mature crRNA:tracrRNA complex that directs the CRISPR nuclease, e.g. Cas9 to the target DNA the target DNA via Watson-Crick base-pairing between the guide sequence portion of the crRNA and the protospacer on the target DNA next to the protospacer adjacent motif (PAM). A skilled artisan will appreciate that each of the engineered RNA molecule of the present invention is further designed such as to associate with a target genomic DNA sequence of interest next to a protospacer adjacent motif (PAM), e.g., a PAM matching the sequence relevant for the type of CRISPR nuclease utilized, such as for a non-limiting example, NGG or NAG, wherein “N” is any nucleobase, for Streptococcus pyogenes Cas9 WT (SpCAS9); NNGRRT for Staphylococcus aureus (SaCas9); NNNVRYM for Jejuni Cas9 WT; NGAN or NGNG for SpCas9-VQR variant; NGCG for SpCas9-VRER variant; NGAG for SpCas9-EQR variant; NRRH for SpCas9-NRRH variant, wherein N is any nucleobase, R is A or G and H is A, C, or T; NRTH for SpCas9-NRTH variant, wherein N is any nucleobase, R is A or G and H is A, C, or T; NRCH for SpCas9-NRCH variant, wherein N is any nucleobase, R is A or G and H is A, C, or T; NG for SpG variant of SpCas9 wherein N is any nucleobase; NG or NA for SpCas9-NG variant of SpCas9 wherein N is any nucleobase; NR or NRN or NYN for SpRY variant of SpCas9, wherein N is any nucleobase, R is A or G and Y is C or T; NNG for Streptococcus canis Cas9 variant (ScCas9), wherein N is any nucleobase; NNNRRT for SaKKH-Cas9 variant of Staphylococcus aureus (SaCas9), wherein N is any nucleobase, and R is A or G; NNNNGATT for Neisseria meningitidis (NmCas9), wherein N is any nucleobase; TTN for Alicyclobacillus acidiphilus Cas12b (AacCas12b), wherein N is any nucleobase; or TTTV for Cpf1, wherein V is A, C or G. RNA molecules of the present invention are each designed to form complexes in conjunction with one or more different CRISPR nucleases and designed to target polynucleotide sequences of interest utilizing one or more different PAM sequences respective to the CRISPR nuclease utilized.

In some embodiments, an RNA-guided DNA nuclease e.g., a CRISPR nuclease, may be used to cause a DNA break, either double or single-stranded in nature, at a desired location in the genome of a cell. The most commonly used RNA-guided DNA nucleases are derived from CRISPR systems, however, other RNA-guided DNA nucleases are also contemplated for use in the genome editing compositions and methods described herein. For instance, see U.S. Patent Publication No. 2015/0211023, incorporated herein by reference.

CRISPR systems that may be used in the practice of the invention vary greatly. CRISPR systems can be a type I, a type II, or a type III system. Non-limiting examples of suitable CRISPR proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas1 Od, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cul966.

In some embodiments, the RNA-guided DNA nuclease is a CRISPR nuclease derived from a type II CRISPR system (e.g., Cas9). The CRISPR nuclease may be derived from Streptococcus pyogenes, Streptococcus thermophilus, Streptococcus sp., Staphylococcus aureus, Neisseria meningitidis, Treponema denticola, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difjicile, Finegoldia magna, Natranaerobius thermophilus, Pelotomaculumthermopropionicum, Acidithiobacillus caldus, Acidithiobacillusferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, or any species which encodes a CRISPR nuclease with a known PAM sequence. CRISPR nucleases encoded by uncultured bacteria may also be used in the context of the invention. (See Burstein et al. Nature, 2017). Variants of CRIPSR proteins having known PAM sequences e.g., SpCas9 D1135E variant, SpCas9 VQR variant, SpCas9 EQR variant, or SpCas9 VRER variant may also be used in the context of the invention.

Thus, an RNA-guided DNA nuclease of a CRISPR system, such as a Cas9 protein or modified Cas9 or homolog or ortholog of Cas9, or other RNA guided DNA nucleases belonging to other types of CRISPR systems, such as Cpf1 and its homologs and orthologs, may be used in the compositions of the present invention. Additional CRISPR nucleases may also be used, for example, the nucleases described in PCT International Application Publication Nos. WO2020/223514 and WO2020/223553, each of which are hereby incorporated by reference.

In certain embodiments, the CRIPSR nuclease may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. Suitable derivatives of a Cas polypeptide or a fragment thereof include but are not limited to mutants, fusions, covalent modifications of Cas protein or a fragment thereof. Derivatives include, but are not limited to, CRISPR nickases, catalytically inactive or “dead” CRISPR nucleases, and fusion of a CRISPR nuclease or derivative thereof to other enzymes such as base editors or retrotransposons. See for example, Anzalone et al. (2019) and PCT International Application No. PCT/US2020/037560.

Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some cases, the cell does not naturally produce Cas protein and is genetically engineered to produce a Cas protein.

In some embodiments, the CRISPR nuclease is Cpf1. Cpf1 is a single RNA-guided endonuclease which utilizes a T-rich protospacer-adjacent motif Cpf1 cleaves DNA via a staggered DNA double-stranded break. Two Cpf1 enzymes from Acidaminococcus and Lachnospiraceae have been shown to carry out efficient genome-editing activity in human cells. (See Zetsche et al., 2015).

Thus, an RNA-guided DNA nuclease of a Type II CRISPR System, such as a Cas9 protein or modified Cas9 or homologs, orthologues, or variants of Cas9, or other RNA guided DNA nucleases belonging to other types of CRISPR systems, such as Cpf1 and its homologs, orthologues, or variants, may be used in the present invention.

In some embodiments, the guide molecule comprises one or more chemical modifications which imparts a new or improved property (e.g., improved stability from degradation, improved hybridization energetics, or improved binding properties with an RNA guided DNA nuclease). Suitable chemical modifications include, but are not limited to: modified bases, modified sugar moieties, or modified inter-nucleoside linkages. Non-limiting examples of suitable chemical modifications include: 4-acetylcytidine, 5-(carboxyhydroxymethyl)uridine, 2′-O-methylcytidine, 5-carboxymethylaminomethyl-2-thiouridine, 5-carboxymethylaminomethyluridine, dihydrouridine, 2′-O-methylpseudouridine, “beta, D-galactosylqueuosine”, 2′-O-methylguanosine, inosine, N6-isopentenyladenosine, 1-methyladenosine, 1-methylpseudouridine, 1-methylguanosine, 1-methylinosine, “2,2-dimethylguanosine”, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, “beta, D-mannosylqueuosine”, 5-methoxycarbonylmethyl-2-thiouridine, 5-methoxycarbonylmethyluridine, 5-methoxyuridine, 2-methylthio-N6-isopentenyladenosine, N-((9-beta-D-ribofuranosyl-2-methylthiopurine-6-yl)carbamoyl)threonine, N-((9-beta-D-ribofuranosylpurine-6-yl)N-methylcarbamoyl)threonine, uridine-5-oxyacetic acid-methylester, uridine-5-oxyacetic acid, wybutoxosine, queuosine, 2-thiocytidine, 5-methyl-2-thiouridine, 2-thiouridine, 4-thiouridine, 5-methyluridine, N-((9-beta-D-ribofuranosylpurine-6-yl)-carbamoyl)threonine, 2′-O-methyl-5-methyluridine, 2-O-methyluridine, wybutosine, “3-(3-amino-3-carboxy-propyl)uridine, (acp3)u”, 2′-0-methyl (M), 3′-phosphorothioate (MS), 3′-thioPACE (MSP), pseudouridine, or 1-methyl pseudo-uridine. Each possibility represents a separate embodiment of the present invention.

Guide Sequences which Target a C3 Allele

Any given RNA molecule comprising a guide sequence portion utilized to target a DNA site may result in degradation of the RNA molecule, limited activity, no activity, or off-target effects. Accordingly, suitable guide sequence portions are necessary for targeting a given DNA site in a gene.

By the present invention, a novel set of guide sequence portions have been identified for targeting at least one C3 allele, introducing to the at least one allele a sequence of nucleotides to be expressed under the control of the C3 promoter, and thereby treating a disorder or disease. Preferably, a non-discriminatory RNA molecule capable of targeting both C3 alleles is used for targeting.

However, RNA molecules capable of targeting a specific C3 allele are also contemplated. For example, a guide sequence portion of an RNA molecule may target a specific SNP present in one C3 allele. Notably, a given gene may contain thousands of SNPs. Utilizing a twenty-five base pair target window for targeting each SNP in a gene would require hundreds of thousands of guide sequences.

The present disclosure provides guide sequence portions capable of specifically targeting a C3 allele. In some embodiments the C3 allele is targeted while leaving the other non-targeted C3 allele unmodified. Some guide sequences of the present invention are designed to, and are most likely to, specifically differentiate between C3 alleles. In some embodiments, both C3 alleles are targeted. Of all possible guide sequences which target a C3 sequence, the specific guide sequences disclosed herein are specifically effective to function with the disclosed embodiments.

Briefly, the guide sequences may have properties as follows: (1) target SNP/insertion/deletion/indel with a high prevalence in the general population, in a specific ethnic population or in a patient population is above 1% and the SNP/insertion/deletion/indel heterozygosity rate in the same population is above 1%; and (2) target a location of a SNP/insertion/deletion/indel proximal to a portion of the gene e.g., within 5 k bases of any portion of the gene, for example, a promoter, a UTR, an exon or an intron. In some embodiments, the prevalence of the SNP/insertion/deletion/indel in the general population, in a specific ethnic population or in a patient population is above 1%, 2%, 3%, 4%, 5%, 6%, 7%, 8%, 9%, 10%, 11%, 12%, 13%, 14%, or 15% and the SNP/insertion/deletion/indel heterozygosity rate in the same population is above 1%, 2%, 3%, 4%, 5%, 6%, 7%, 8%, 9%, 10%, 11%, 12%, 13%, 14%, or 15%. Each possibility represents a separate embodiment and may be combined at will.

In some embodiments of the present invention, an RNA molecule is used to target a site in the C3 gene to introduce, or knock-in, an exogenous sequence of nucleotides into the C3 gene. In some embodiments, the site is a SNP position. In some embodiments, the location of the site is near the intended knock-in site, preferably near the start codon or the stop codon, preferably within 150 basepairs of the start codon or stop codon. In some embodiments, the site is located within the first intron that follows the first coding exon of a targeted C3 allele or alleles. In some embodiments, the site is located within intron 1 of a targeted C3 allele.

A guide sequence portion of the present invention may: (1) target a heterozygous SNP for the targeted C3 gene; (2) target a position (common sequence or a SNP) upstream or downstream of the gene; (3) target a SNP with a prevalence of the SNP/insertion/deletion/indel in the general population, in a specific ethnic population, or in a patient population above 1%; (4) have a guanine-cytosine content of greater than 30% and less than 85%; (5) have no repeat of seven or more thymine/uracil, guanine, cytosine, or adenine; and (6) have no additional target in the genome with zero mismatch with the same PAM sequence. Guide sequences of the present invention may satisfy any one of the above criteria and are most likely to differentiate between a targeted allele from its corresponding non-targeted allele.

### Delivery to Cells

The compositions described herein may be delivered to a target cell by any suitable means. Compositions of the present invention may be targeted to any cell which contains and/or expresses a C3 allele, including any mammalian cell, preferably a liver cell. For example, in one embodiment an RNA molecule that specifically targets a C3 allele is delivered to a target cell and the target cell is an HSC, hepatocyte, or other liver cell. The delivery to the cell may be performed in-vitro, ex-vivo, or in-vivo. Further, the nucleic acid compositions described herein may be delivered as one or more of DNA molecules, RNA molecules, ribonucleoproteins (RNPs), nucleic acid vectors, or any combination thereof.

In some embodiments, in vivo delivery methods of the compositions described herein include delivery by a lentivirus, adeno-associated virus (AAV) or nanoparticle. In some embodiments, in vivo delivery methods of the compositions described herein include delivery by a lentivirus, adeno-associated virus (AAV) or nanoparticle. The composition may be in the form of an RNP composition. Accordingly, the delivery can be in vivo to liver cells within a subject, for example, a subject suffering from a liver-related disorder.

In some embodiments, any one of the compositions described herein is delivered to a cell ex-vivo. In some embodiments, the cell is a stem cell. In some embodiments, the cell is a liver cell. In some embodiments, the cell is a hepatocyte. In some embodiments, the cell is an iPS-derived hepatocyte. The composition may be delivered to the cell by any known ex-vivo delivery method, including but not limited to, electroporation, viral transduction, nanoparticle delivery, liposomes, etc. The composition may be in the form of an RNP composition. Additional detailed delivery methods are described throughout this section.

In some embodiments, option a liver lobe or hepatocyte cells are obtained from a subject, for example, by performing a biopsy. The obtained cells may then be genetically modified by ex vivo delivery of a composition described herein. The modified cells may be re-introduced to the subject by performing a lobe transplantation or hepatocyte transplantation.

In some embodiments, an RNA molecule of a composition described herein comprises a chemical modification. Non-limiting examples of suitable chemical modifications include 2′-0-methyl (M), 2′-0-methyl, 3′phosphorothioate (MS) or 2′-0-methyl, 3′thioPACE (MSP), pseudouridine, and 1-methyl pseudo-uridine. Each possibility represents a separate embodiment of the present invention.

Any suitable viral vector system may be used to deliver nucleic acid compositions e.g., the RNA molecule compositions of the subject invention. Conventional viral and non-viral based gene transfer methods can be used to introduce nucleic acids and target tissues. In certain embodiments, nucleic acids are administered for in vivo or ex vivo gene therapy uses. Non-viral vector delivery systems include naked nucleic acid, and nucleic acid complexed with a delivery vehicle such as a liposome or poloxamer. For a review of gene therapy procedures, see Anderson (1992); Nabel & Felgner (1993); Mitani & Caskey (1993); Dillon (1993); Miller (1992); Van Brunt (1988); Vigne (1995); Kremer & Perricaudet (1995); Haddada et al. (1995); and Yu et al. (1994).

Methods of non-viral delivery of nucleic acids and/or proteins include electroporation, lipofection, microinjection, biolistics, particle gun acceleration, virosomes, liposomes, immunoliposomes, lipid nanoparticles (LNPs), polycation or lipid:nucleic acid conjugates, artificial virions, and agent-enhanced uptake of nucleic acids or can be delivered to plant cells by bacteria or viruses (e.g., Agrobacterium, Rhizobium sp. NGR234, Sinorhizoboiummeliloti, Mesorhizobium loti, tobacco mosaic virus, potato virus X, cauliflower mosaic virus and cassava vein mosaic virus). (See, e.g., Chung et al., 2006). Sonoporation using, e.g., the Sonitron 2000 system (Rich-Mar), can also be used for delivery of nucleic acids. Cationic-lipid mediated delivery of proteins and/or nucleic acids is also contemplated as an in vivo, ex vivo, or in vitro delivery method. (See Zuris et al. (2015); see also Coelho et al. (2013); Judge et al. (2006); and Basha et al. (2011)).

Non-viral vectors, such as transposon-based systems e.g. recombinant Sleeping Beauty transposon systems or recombinant PiggyBac transposon systems, may also be delivered to a target cell and utilized for transposition of a polynucleotide sequence of a molecule of the composition or a polynucleotide sequence encoding a molecule of the composition in the target cell.

Additional exemplary nucleic acid delivery systems include those provided by Amaxa.RTM. Biosystems (Cologne, Germany), Maxcyte, Inc. (Rockville, Md.), BTX Molecular Delivery Systems (Holliston, Mass.) and Copernicus Therapeutics Inc., (see, e.g., U.S. Pat. No. 6,008,336). Lipofection is described in e.g., U.S. Pat. Nos. 5,049,386, 4,946,787; and 4,897,355, and lipofection reagents are sold commercially (e.g., Transfectam.TM., Lipofectin.TM. and Lipofectamine.TM. RNAiMAX). Cationic and neutral lipids that are suitable for efficient receptor-recognition lipofection of polynucleotides include those disclosed in PCT International Publication Nos. WO/1991/017424 and WO/1991/016024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science (1995); Blaese et al., (1995); Behr et al., (1994); Remy et al. (1994); Gao and Huang (1995); Ahmad and Allen (1992); U.S. Pat. Nos. 4,186,183; 4,217,344; 4,235,871; 4,261,975; 4,485,054; 4,501,728; 4,774,085; 4,837,028; and 4,946,787).

Additional methods of delivery include the use of packaging the nucleic acids to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (See MacDiarmid et al., 2009).

Delivery vehicles include, but are not limited to, bacteria, preferably non-pathogenic, vehicles, nanoparticles, exosomes, microvesicles, gene gun delivery, for example, by attachment of a composition to a gold particle which is fired into a cell using via a “gene-gun”, viral vehicles, including but not limited to lentiviruses, AAV, and retroviruses), virus-like particles (VLPs). large VLPs (LVLPs), lentivirus-like particles, transposons, viral vectors, naked vectors, DNA, or RNA, among other delivery vehicles known in the art.

The delivery of a CRISPR nuclease and/or a polynucleotide encoding the CRIPSR nuclease, and optionally additional nucleotide molecules and/or additional proteins or peptides, may be performed by utilizing a single delivery vehicle or method or a combination of different delivery vehicles or methods. For example, a CRISPR nuclease may be delivered to a cell utilizing an LNP, and a crRNA molecule and tracrRNA molecule may be delivered to the cell utilizing AAV. Alternatively, a CRISPR nuclease may be delivered to a cell utilizing an AAV particle, and a crRNA molecule and tracrRNA molecule may be delivered to the cell utilizing a separate AAV particle, which may be advantageous due to size limitations.

The use of RNA or DNA viral based systems for viral mediated delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids include, but are not limited to, retroviral, lentivirus, adenoviral, adeno-associated, vaccinia and herpes simplex virus vectors for gene transfer. An RNA virus is may be utilized for delivery of the RNA compositions described herein. Additionally, high transduction efficiencies have been observed in many different cell types and target tissues. Nucleic acid of the invention may be delivered by non-integrating lentivirus. Optionally, RNA delivery with Lentivirus is utilized. Optionally the lentivirus includes mRNA of the nuclease, RNA of the guide. Optionally the lentivirus includes mRNA of the nuclease, RNA of the guide and a donor template. Optionally, the lentivirus includes the nuclease protein, guide RNA. Optionally, the lentivirus includes the nuclease protein, guide RNA and/or a donor template for gene editing via, for example, homology directed repair. Optionally the lentivirus includes mRNA of the nuclease, DNA-targeting RNA, and the tracrRNA. Optionally the lentivirus includes mRNA of the nuclease, DNA-targeting RNA, and the tracrRNA, and a donor template. Optionally, the lentivirus includes the nuclease protein, DNA-targeting RNA, and the tracrRNA. Optionally, the lentivirus includes the nuclease protein, DNA-targeting RNA, and the tracrRNA, and a donor template for gene editing via, for example, homology directed repair.

As mentioned above, the compositions described herein may be delivered to a target cell using a non-integrating lentiviral particle method, e.g. a LentiFlash® system. Such a method may be used to deliver mRNA or other types of RNAs into the target cell, such that delivery of the RNAs to the target cell results in assembly of the compositions described herein inside of the target cell. See also PCT International Publication Nos. WO/2013/014537, WO/2014/016690, WO/2016/185125, WO/2017/194902, and WO/2017/194903.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof (See, e.g., Buchschacher et al. (1992); Johann et al. (1992); Sommerfelt et al. (1990); Wilson et al. (1989); Miller et al. (1991); PCT International Publication No. WO/1994/026877A1).

At least six viral vector approaches are currently available for gene transfer in clinical trials, which utilize approaches that involve complementation of defective vectors by genes inserted into helper cell lines to generate the transducing agent.

pLASN and MFG-S are examples of retroviral vectors that have been used in clinical trials (See Dunbar et al., 1995; Kohn et al., 1995; Malech et al., 1997). PA317/pLASN was the first therapeutic vector used in a gene therapy trial (Blaese et al., 1995). Transduction efficiencies of 50% or greater have been observed for MFG-S packaged vectors. (Ellem et al., (1997); Dranoff et al., 1997).

Packaging cells are used to form virus particles that are capable of infecting a host cell. Such cells include 293 cells, which package adenovirus, AAV, and Psi-2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additionally, AAV can be produced at clinical scale using baculovirus systems (see U.S. Pat. No. 7,479,554).

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al. (1995) reported that Moloney murine leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences which favor uptake by specific target cells.

Gene therapy vectors can be delivered in vivo by administration to an individual patient, typically by systemic administration (e.g., intravitreal, intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, optionally after selection for cells which have incorporated the vector. A non-limiting exemplary ex vivo approach may involve removal of tissue (e.g., peripheral blood, bone marrow, and spleen) from a patient for culture, nucleic acid transfer to the cultured cells (e.g., hematopoietic stem cells), followed by grafting the cells to a target tissue (e.g., bone marrow, and spleen) of the patient. In some embodiments, the stem cell or hematopoietic stem cell may be further treated with a viability enhancer.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a nucleic acid composition, and re-infused back into the subject organism (e.g., patient). Various cell types suitable for ex vivo transfection are well known to those of skill in the art (See, e.g., Freshney, “Culture of Animal Cells, A Manual of Basic Technique and Specialized Applications (6th edition, 2010) and the references cited therein for a discussion of how to isolate and culture cells from patients).

Suitable cells include, but are not limited to, eukaryotic cells and/or cell lines. Non-limiting examples of such cells or cell lines generated from such cells include COS, CHO (e.g., CHO—S, CHO-K1, CHO-DG44, CHO-DUXB11, CHO-DUKX, CHOK1SV), VERO, MDCK, WI38, V79, B14AF28-G3, BHK, HaK, NSO, SP2/0-Ag14, HeLa, HEK293 (e.g., HEK293-F, HEK293-H, HEK293-T), perC6 cells, any plant cell (differentiated or undifferentiated), as well as insect cells such as Spodopterafugiperda (Sf), or fungal cells such as Saccharomyces, Pichia and Schizosaccharomyces. In certain embodiments, the cell line is a CHO-K1, MDCK or HEK293 cell line. Additionally, primary cells may be isolated and used ex vivo for reintroduction into the subject to be treated following treatment with a guided nuclease system (e.g. CRISPR/Cas). Suitable primary cells include peripheral blood mononuclear cells (PBMC), and other blood cell subsets such as, but not limited to, CD4+ T cells or CD8+ T cells. Suitable cells also include stem cells such as, by way of example, embryonic stem cells, induced pluripotent stem cells, hematopoietic stem cells (CD34+), neuronal stem cells and mesenchymal stem cells.

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-gamma, and TNF-alpha are known (as a non-limiting example see, Inaba et al., 1992).

Stem cells are isolated for transduction and differentiation using known methods. For example, stem cells are isolated from bone marrow cells by panning the bone marrow cells with antibodies which bind unwanted cells, such as CD4+ and CD8+(T cells), CD45+(panB cells), GR-1 (granulocytes), and lad (differentiated antigen presenting cells) (as a non-limiting example, see Inaba et al., 1992). Stem cells that have been modified may also be used in some embodiments.

Vectors (e.g., retroviruses, liposomes, etc.) containing therapeutic nucleic acid compositions can also be administered directly to an organism for transduction of cells in vivo. Administration is by any of the routes normally used for introducing a molecule into ultimate contact with blood or tissue cells including, but not limited to, injection, infusion, topical application (e.g., eye drops and cream) and electroporation. Suitable methods of administering such nucleic acids are available and well known to those of skill in the art, and, although more than one route can be used to administer a particular composition, a particular route can often provide a more immediate and more effective reaction than another route. According to some embodiments, the composition is delivered via IV injection.

Vectors suitable for introduction of transgenes into immune cells (e.g., T-cells) include non-integrating lentivirus vectors. See, e.g., U.S. Patent Publication No. 2009/0117617.

Pharmaceutically acceptable carriers are determined in part by the particular composition being administered, as well as by the particular method used to administer the composition. Accordingly, there is a wide variety of suitable formulations of pharmaceutical compositions available, as described below (See, e.g., Remington's Pharmaceutical Sciences, 17th ed., 1989).

In accordance with some embodiments, there is provided an RNA molecule which binds to/associates with and/or directs the RNA guided DNA nuclease to a sequence comprising at least one nucleotide which differs between alleles (e.g., SNP) of a gene of interest (i.e., a sequence of one C3 allele which is not present in the other C3 allele). Any sequence difference between C3 alleles may be targeted by an RNA molecule of the present invention to modify or edit a single C3 allele.

The disclosed compositions and methods may also be used in the manufacture of a medicament for treating dominant genetic disorders in a patient.

### Mechanisms of Action for C3 Safe Harbor Knock-In Methods

Without being bound by any theory or mechanism, the instant invention may be utilized to apply a CRISPR nuclease to process a C3 allele in order to introduce a sequence to a safe harbor site within the C3 allele and thereby control expression of the introduced sequence by the promoter of the C3 allele. Expression of the introduced sequence thereby prevents or treats a liver-related disorder. A specific guide sequence may be selected from Table 1 based on the targeted position and the type of CRISPR nuclease used (e.g. according to a required PAM sequence).

The C3 gene is located on chromosome 19 and encodes the Complement component 3 protein. A donor molecule may be used to introduce a desired sequence of nucleotides into a C3 safe harbor site via knock-in. Accordingly, any sequence encoding an expression product that prevents or treats a liver-related disease may be introduced at a C3 safe harbor site. Accordingly, non-limiting examples of sequences encoded by a donor molecule include, ATP7B, A1AT, G6PC, SERPINA, LDLR, TTR, OTC—omithine transcarbamylase, ASD—argininosuccinic acid synthetase, arginase, argininosuccinase, carbamoyl phosphate synthetase, and N-acetylglutamate synthetase, or portions thereof.

One strategy is to knock-in a sequence of nucleotides in the first intron or the first intron that follows the first coding exon of the C3 gene. This strategy utilizes an RNA molecule to target a CRISPR nuclease to Intron 1 of the C3 gene and thereby mediate a double-stranded break. Since the break is mediated in a nonregulatory region, it is not expected to affect the expression of the gene. For knock-in of a sequence without a knockout of C3, the sequence is inserted as a new exon (namely, Exon 2), by adding splicing acceptor (SA) and splicing donor (SD) elements to the knock-in donor cassette. In addition, to prevent the interruption of the expression of the C3 gene, the donor cassette includes at least one 2A self-cleaving peptide and/or the signal peptide of C3 at the C-terminus of the inserted gene, which is cleaved in the endoplasmic reticulum and enables the separation of the inserted sequence protein expression product from C3 protein.

Another strategy is to knock-in a sequence of nucleotides in the first intron of a specific C3 allele. According to the gnomAD, there are healthy individuals with C3 frameshift mutations, which suggests that haploinsufficiency is not a concern. Therefore, the knocked-in sequence of nucleotides can be inserted into a single C3 allele as a new exon with a translation termination signal, thereby knocking-out the expression of only one C3 allele while the other C3 allele remains unaffected. A C3 allele-specific editing event can be achieved, for example, by targeting a highly frequent SNP such as rs199713383 located in Intron 1.

Another strategy is to knock-in a sequence of nucleotides in the C3 gene by replacing the stop codon. In this case a biallelic break will be mediated in a 3′UTR region either up to 150 nucleotides downstream of the stop codon or upstream to the stop codon but in an intron region. The location of the break in these regions does not affect the expression of C3. The knocked-in sequence of nucleotides is inserted in place of the stop codon and the knock-in donor cassette contains at least one 2A self-cleaving peptide at the N-terminus of the inserted sequence of nucleotides enabling the separation of the inserted sequence protein expression product from C3 protein.

Another strategy is to knock-in a sequence of nucleotides in a specific C3 allele by replacing the stop codon. This can be achieved by utilizing an RNA molecule to target a CRISPR nuclease to a SNP position located within 150 nucleotides upstream to the stop codon, such as rs17030 located in Exon 41 of a C3 allele.

Examples of RNA Guide Sequences which Specifically Target Alleles of the C3 Gene

Although a large number of guide sequences can be designed to target a C3 allele, the nucleotide sequences described in Table 1 identified by SEQ ID NOs: 1-7,046 below were specifically selected to effectively implement the methods set forth herein and to effectively discriminate between alleles.

Table 1 shows guide sequences designed for use as described in the embodiments above to associate with C3 alleles. Each engineered guide molecule is further designed such as to associate with a target genomic DNA sequence of interest that lies next to a protospacer adjacent motif (PAM), e.g., a PAM matching the sequence NGG or NAG, where “N” is any nucleobase. The guide sequences were designed to work in conjunction with one or more different CRISPR nucleases, including, but not limited to, e.g. SpCas9WT (PAM SEQ: NGG), SpCas9.VQR.1 (PAM SEQ: NGAN), SpCas9.VQR.2 (PAM SEQ: NGNG), SpCas9.EQR (PAM SEQ: NGAG), SpCas9.VRER (PAM SEQ: NGCG), SaCas9WT (PAM SEQ: NNGRRT), SpRY (PAM SEQ: NRN or NYN), NmCas9WT (PAM SEQ: NNNNGATT), Cpf1 (PAM SEQ: TTTV), or JeCas9WT (PAM SEQ: NNNVRYM). RNA molecules of the present invention are each designed to form complexes in conjunction with one or more different CRISPR nucleases and designed to target polynucleotide sequences of interest utilizing one or more different PAM sequences respective to the CRISPR nuclease utilized.

TABLE 1
Guide sequence portions designed to associate with specific C3 gene targets

| | SEQ ID NOs: of | SEQ ID NOs: of | SEQ ID NOs: of |
|---|---|---|---|
| | 20-nucleotide guide | 21-nucleotide guide | 22-nucleotide guide |
| Target | sequence portions | sequence portions | sequence portions |
| 19:6677978_G_A | 1-49 | 50-101 | 102-155 |
| rs17030_REF | | | |
| 19:6677978_G_A | 1, 3, 5, 8, 16, 20, 23, | 50-51, 55, 58, 66, 70, | 102-103, 107, 118, |
| rs17030_SNP | 28, 35, 41, 47, 156- | 73, 78, 85, 91-92, 99, | 121, 123, 126-127, |
| | 192 | 193-232 | 132, 145-146, 153, |
| | | | 233-274 |
| 19:6720063_A_AT | 275-324 | 325-376 | 377-430 |
| rs199713383_REF | | | |
| 19:6720063_A_AT | 280, 282, 285, 287- | 327, 330, 332, 335, | 379, 382, 384, 387, |
| rs199713383_SNP | 288, 290, 306, 312, | 338, 340, 356, 364, | 390, 409, 413, 416- |
| | 314, 317, 323, 431-468 | 366, 374-375, 469-508 | 417, 419, 428, 509-550 |
| 19:6720065_AC_A | 275-276, 278-279, | 325-326, 328-361, | 377-407, 409-414, |
| rs35473940_REF | 281-310, 312-320, | 364-372, 374-376, | 417-426, 428-430, |
| | 322-324, 551-554 | 555-558 | 559-562 |
| 19:6720065_AC_A | 276, 279, 281, 288, | 326, 329, 331, 337, | 378, 381, 383, 389, |
| rs35473940_SNP | 292, 296, 312, 315, | 342, 346, 360, 364, | 392, 394, 413, 426, |
| | 322, 553-554, 563-592 | 374, 557-558, 593-624 | 428, 561-562, 625-658 |
| 19:6677732-6677881 | 659-908 | 909-1156 | 1157-1402 |
| 3′UTR | | | |
| 19:6719404-6720515 | 275-324, 551-554, | 325-376, 555-558, | 377-430, 559-562, |
| Intron 1 | 583, 1403-3289 | 611, 3290-5153 | 656, 5154-7046 |

The indicated locations listed in column 1 of the Table 1 are based on gnomAD v3 database and UCSC Genome Browser assembly ID: hg38, Sequencing/Assembly provider ID: Genome Reference Consortium Human GRCh38.p12 (GCA_000001405.27). Assembly date: December 2013 initial release; Dececember 2017 patch release 12.
The SNP details are indicated by the listed SNP ID Nos. (“rs numbers”), which are based on the NCBI 2018 database of Single Nucleotide Polymorphisms (dbSNP)). The indicated DNA mutations are associated with Transcript Consequence NM_001256054 as obtained from NCBI RefSeq genes.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

### EXPERIMENTAL DETAILS

### Example 1: C3 On-Target Activity Analysis

Guide sequences comprising 17-50 contiguous nucleotides containing nucleotides in the sequence set forth in any one of SEQ ID NOs: 1-7,046 are screened for high on target activity using SpCas9 in HeLa cells. On target activity is determined by DNA capillary electrophoresis analysis.

### Example 2: Activity of C3-Targeting RNA Guide Molecules

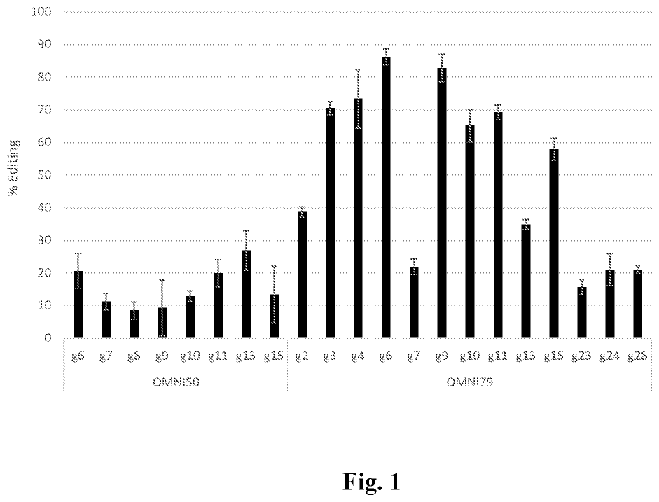

The C3 gene was selected as a genomic safe harbor site to target in liver cells due to its abundant expression in the target tissue and its ability to accommodate the integration of new sequences in a manner that ensures secretion and preserves C3 expression. The insertion site was engineered into the first intron of C3, in close proximity to a secretory peptide encoded at the first exon and ultimately cleaved from the final protein product. A guide screen using OMNI-50 and OMNI-79 CRISPR nucleases was performed in HeLa cells. Briefly, an OMNI-50 or OMNI-79 nuclease encoding plasmid (6 4ng) was co-transfected with each of the guide-encoding DNA plasmids (2 0ng) in a 96 well plate format using jetOPTIMUS reagent (Polyplus). Cells were harvested 72 hours post-DNA transfection, genomic DNA was extracted, and then used for next-generation sequencing (NGS) analyses. The graph in FIG. 1 represents the average of % editing ±standard deviation (STDV) of three (3) independent experiments. NGS analyses shows a range of 8% to 86% editing for both nucleases. Notably, OMNI-50 RNP compositions generally exhibit increased activity compared to DNA-based compositions, therefore DNA screening in HeLa cells has lower cut-off for proof of concept.

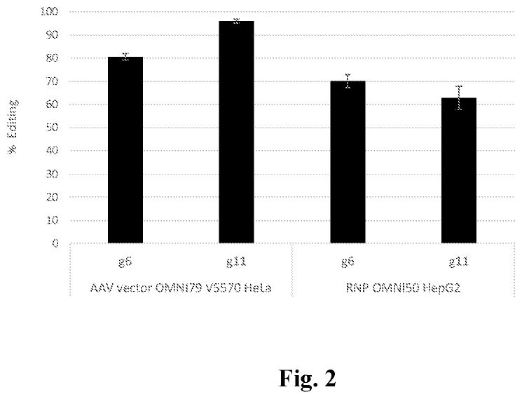

The CRISPR nuclease and the HDR template can be delivered to the target cells by adeno-associated virus (AAV). Due to its small size, OMNI-79 nuclease can be packed in AAV together with a guide molecule. Guides “g6” and “g11” were cloned into a AAV vector with OMNI-79 V5570 nuclease to test activity in HeLa cells (FIG. 2). Briefly, AAV plasmid (15 0ng) was transfected in a 96 well plate format using jetOPTIMUS reagent (Polyplus). Genomic DNA was extracted 72 hours post-transfection and analyzed by NGS. The graph in FIG. 2 represents the average of % editing ±STDV of three (3) independent experiments.

Activity of OMNI-50 guides “g6” and “g11” was evaluated in HepG2 cells by RNP electroporation. Briefly, 4×10⁵ HepG2 cells were mixed with preassembled RNPs composed of 105 pmole OMNI-50 protein and 120 pmole sgRNAs, and then mixed with 100 pmole of an electroporation enhancer (IDT-1075916). The mix was then electroporated into the cells using SF Cell 4D-Nucleofector X Kit S (PBC2-00675, Lonza) by applying the DS-123 program. A fraction of the cells were harvested 72 hours post-electroporation and genomic DNA was extracted to measure on-target activity by NGS. According to NGS analysis, both guides demonstrated high indel formation activity (FIG. 2).

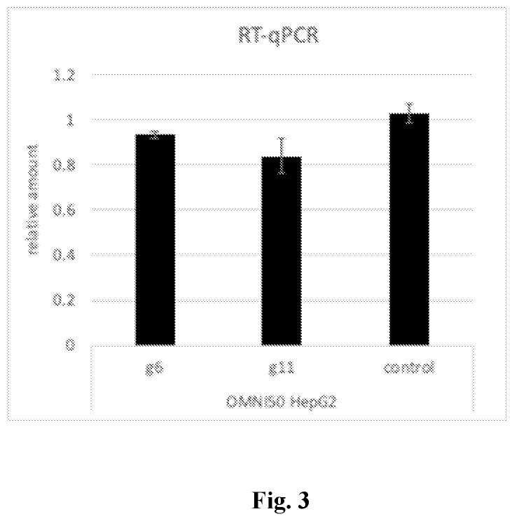

To assess the effect of OMNI-50 guide composition editing on C3 expression, total RNA was extracted from HepG2 cells seven (7) days post-electroporation and the mRNA levels of C3 was measured by qRT-PCR. The results demonstrated preserved expression of C3 since only a minor effect on the level of C3 mRNA was detected (FIG. 3).

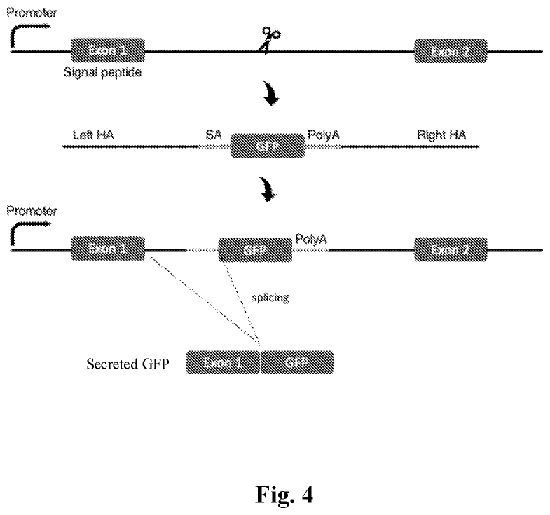

To validate the feasibility of HDR at a C3 locus, three constructs with homology arm lengths of 500 nucleotides (SEQ ID NO: 7054), 800 nucleotides (SEQ ID NO: 7053), and 927 nucleotides (SEQ ID NO: 7052) were cloned. Homology arms were positioned at the cut site of a “g11” guide molecule. A sequence encoding GFP with a poly-A tail was used as a reporter gene in the HDR template. Splice acceptor sites 32 nucleotides in length were added upstream of the GFP sequence (FIG. 4). In this case, the splice site sequences was taken from a pmaxGFP vector (Lonza), although other splice sequences could be used. This specific HDR template insertion was used as a proof of concept and interferes with C3 endogenous protein expression. Other donor templates could be designed with a P2A peptide sequences in order to avoid the use of a “STOP” codon and allow C3 protein expression.

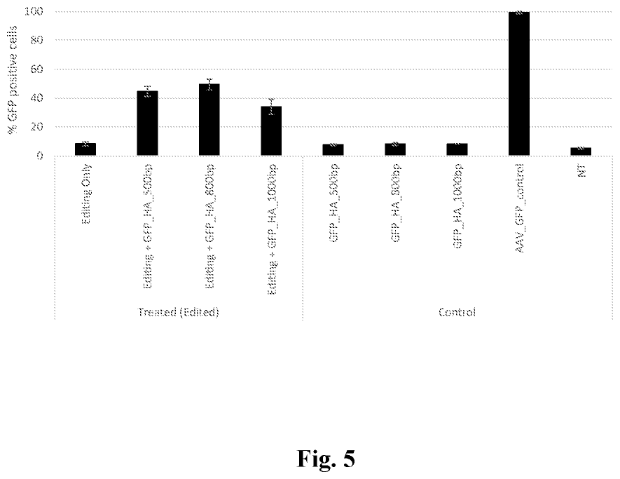

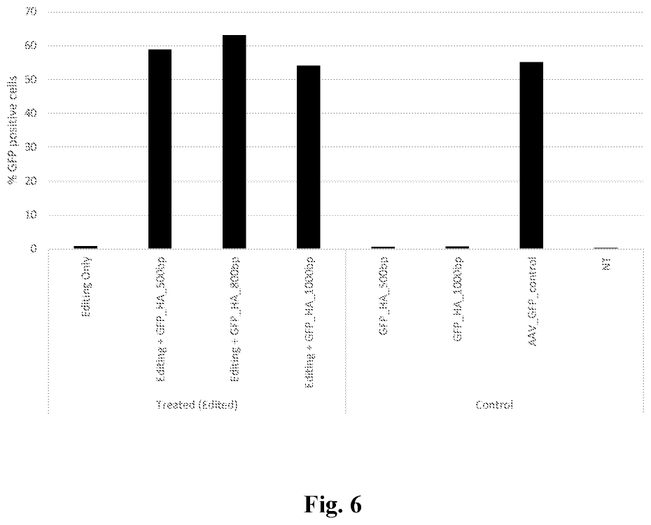

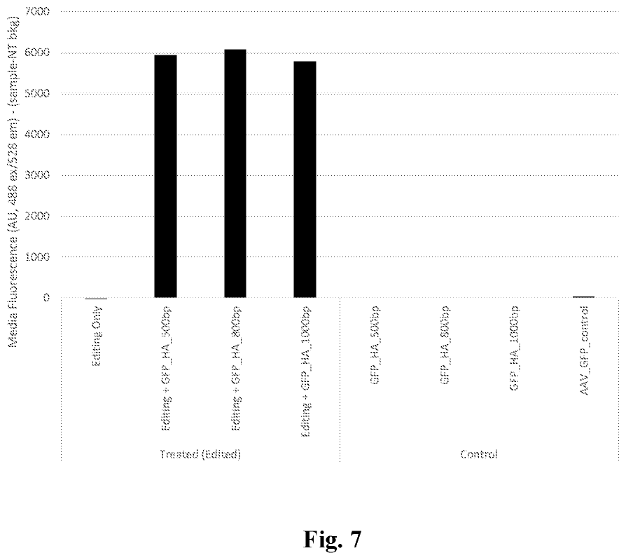

OMNI-79 V5570 nuclease and C3-targeting “g11 ” guide molecule, or a GFP HDR template, were packed in AAV DJ viral particles. HepG2 cells were infected with each of the indicated AAV particles at a multiplicity of infection (MOI) of 10⁵. Cells were analyzed by flow cytometry for GFP expression 72 hours and 15 days following treatment (FIG. 5 and FIG. 6). GFP secretion was evaluated by a plate reader (FIG. 7). Editing levels were analyzed after 72 hours by NGS after DNA extraction. Cells infected with the nuclease and guide molecule alone showed editing of 42.24±4.4%. There is a correlation between editing levels and HDR rates indicating that HDR is very efficient.

GFP fluorescence was also detected in the cell media (FIG. 7), indicating correct integration and splicing of the HDR template.

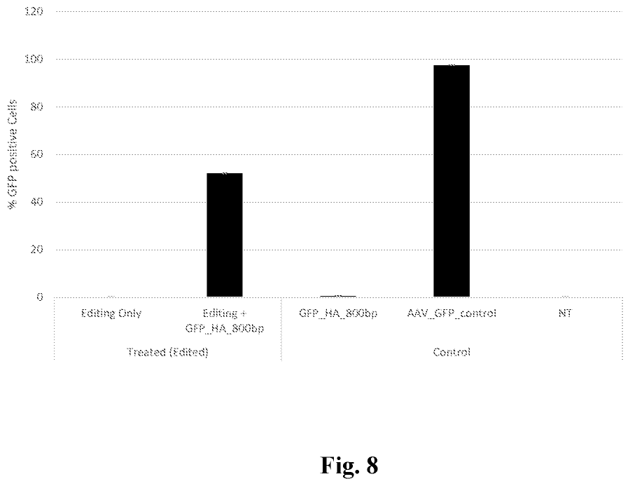

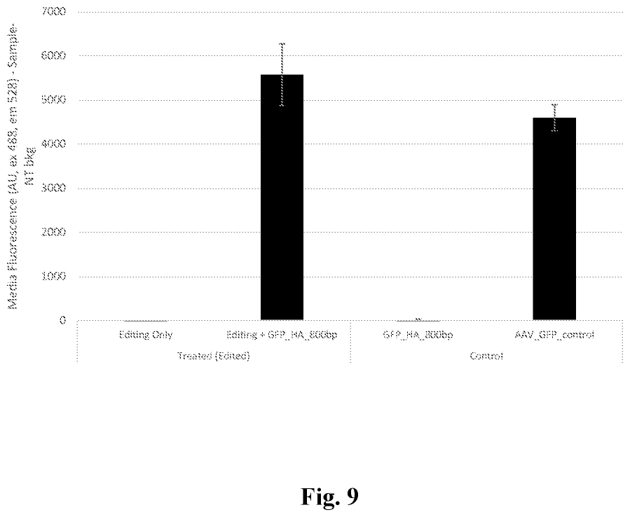

Similar HDR levels were also demonstrated by RNP editing with OMNI-50 nuclease and a C3-targeting “g11 ” guide using electroporation and AAV delivery of an HDR template (FIG. 8, FIG. 9). Editing levels were 53.6±1.05% in the RNP only treated cells.

TABLE 2
22-nucletoide guide sequence portions
targeting intron 1 of C3.

| Guide Name | Sequence | PAM |
|---|---|---|
| g2 | GGGUUUAAAGAUUGGCUACUUC | GGG |
| | (SEQ ID NO: 6417) | |
| g3 | UACUUCGGGUUCAUGUAUAGUU | UGG |
| | (SEQ ID NO: 6617) | |
| g4 | ACUUCGGGUUCAUGUAUAGUUG | GGG |
| | (SEQ ID NO: 5403) | |
| g6 | AGGUUUUGGCGUAUGUGGGUUU | GGG |
| | (SEQ ID NO: 5493) | |
| g7 | UUUGGCGUAUGUGGGUUUGGGU | AGG |
| | (SEQ ID NO: 7030) | |
| g8 | UGGGUUUGGGUAGGUAGAGUUG | AGG |
| | (SEQ ID NO: 6873) | |
| g9 | GGGUUUGGGUAGGUAGAGUUGA | GGG |
| | (SEQ ID NO: 6421) | |
| g10 | GGUAGGUAGAGUUGAGGGUGUC | CGG |
| | (SEQ ID NO: 6425) | |
| g11 | GUAGGUAGAGUUGAGGGUGUCC | GGG |
| | (SEQ ID NO: 6473) | |
| g13 | GCCAGGCCCAGCUGGCUGAGAG | UGG |
| | (SEQ ID NO: 6240) | |
| g15 | AGCUGGCUGAGAGUGGGAGUCA | UGG |
| | (SEQ ID NO: 5453) | |
| g23 | GAGGUUUGGACUUUACAAGAUU | UGG |
| | (SEQ ID NO: 6176) | |
| g24 | AGGUUUGGACUUUACAAGAUUU | GGG |
| | (SEQ ID NO: 5492) | |
| g28 | GUAUUUAUUAGGACUGGGGUGU | UGG |
| | (SEQ ID NO: 6481) | |

TABLE 3
OMNI CRISPR nuclease sequences, PAM
requirements, and sgRNA scaffold
sequences

| OMNI CRISPR | | |
|---|---|---|
| Nuclease and | PAM | sgRNA Scaffold |
| gRNA | Sequence | Sequence |
| OMNI-79 | NGR | GUUGCCGCUGGAGAAAUC |
| (SEQ ID | | CAGUUGUUAACAAGCAGC |
| NO: 7055) | | UUGACUGCACCAAAUAAG |
| | | GCGGGGGCUGCGGCCCUC |
| | | GCUUUUUU |
| | | (SEQ ID NO: 7058) |
| OMNI-79 V5570 | NGR | GUUGCCGCUGGAGAAAUC |
| (SEQ ID | | CAGUUGUUAACAAGCAGC |
| NO: 7056) | | UUGACUGCACCAAAUAAG |
| | | GCGGGGGCUGCGGCCCUC |
| | | GCUUUUUU |
| | | (SEQ ID NO: 7058) |
| OMNI-50 | NGG | GUUUGAGAGUUAUGUAAG |
| (SEQ ID | | AAAUUACAUGACGAGUUC |
| NO: 7057) | | AAAUAAAAAUUUAUUCAA |
| | | ACCGCCUAUUUAUAGGCC |
| | | GCAGAUGUUCUGCAUUAU |
| | | GCUUGCUAUUGCAAGCUU |
| | | UUUU |
| | | (SEQ ID NO: 7059) |

OMNI-79 CRISPR nuclease and OMNI-50 CRISPR nuclease are described in PCT International Application Publication Nos. WO/2021/248016 and WO/2020/223513, respectively, the contents of each of which are hereby incorporated by reference.

### REFERENCES

1. Ahmad and Allen (1992) “Antibody-mediated Specific Binging and Cytotoxicity of Lipsome-entrapped Doxorubicin to Lung Cancer Cells in Vitro”, Cancer Research 52:4817-20.
2. Anderson (1992) “Human gene therapy”, Science 256:808-13.
3. Anzalone et al. (2019) “Search-and-replace genomme editing without double-strand brakes or donor DNA”, Nature 576, 149-157.
4. Basha et al. (2011) “Influence of Cationic Lipid Composition on Gene Silencing Properties of Lipid Nanoparticle Formulations of siRNA in Antigen-Presenting Cells”, Mol. Ther. 19(12):2186-200.
5. Behr (1994) Gene transfer with synthetic cationic amphiphiles: Prospects for gene therapy”, Bioconjuage Chem 5:382-89.
6. Blaese (1995) “Vectors in cancer therapy: how will they deliver”, Cancer Gene Ther. 2:291-97.
7. Blaese et al. (1995) “T lymphocyte-directed gene therapy for ADA-SCID: initial trial results after 4 years”, Science 270(5235):475-80.
8. Buchschacher and Panganiban (1992) “Human immunodeficiency virus vectors for inducible expression of foreign genes”, J. Virol. 66:2731-39.
9. Burstein et al. (2017) “New CRISPR-Cas systems from uncultivated microbes”, Nature 542:237-41.
10. Chang and Wilson (1987) “Modification of DNA ends can decrease end-joining relative to homologous recombination in mammalian cells”, Proc. Natl. Acad. Sci. USA 84:4959-4963.
11. Chung et al. (2006) “Agrobacterium is not alone: gene transfer to plants by viruses and other bacteria”, Trends Plant Sci. 11(1):1-4.
12. Coelho et al. (2013) “Safety and efficacy of RNAi therapy for transthyretin amyloidosis” N. Engl. J. Med. 369, 819-829.
13. Collias and Beisel (2021) “CRISPR technologies and the search for the PAM-free nuclease”, Nature Communications 12, 555.
14. Crystal (1995) “Transfer of genes to humans: early lessons and obstacles to success”, Science 270(5235):404-10.
15. Dillon (1993) “Regulation gene expression in gene therapy” Trends in Biotechnology 11(5):167-173.
16. Dranoff et al. (1997) “A phase I study of vaccination with autologous, irradiated melanoma cells engineered to secrete human granulocyte macrophage colony stimulating factor”, Hum. Gene Ther. 8(1):111-23.
17. Dunbar et al. (1995) “Retrovirally marked CD34-enriched peripheral blood and bone marrow cells contribute to long-term engraftment after autologous transplantation”, Blood 85:3048-57.
18. Ellem et al. (1997) “A case report: immune responses and clinical course of the first human use of granulocyte/macrophage-colony-stimulating-factor-transduced autologous melanoma cells for immunotherapy”, Cancer Immunol Immunother 44:10-20.
19. Gao and Huang (1995) “Cationic liposome-mediated gene transfer” Gene Ther. 2(10):710-22.
20. Haddada et al. (1995) “Gene Therapy Using Adenovirus Vectors”, in: The Molecular Repertoire of Adenoviruses III: Biology and Pathogenesis, ed. Doerfler and Bohm, pp. 297-306.
21. Han et al. (1995) “Ligand-directed retro-viral targeting of human breast cancer cells”, Proc Natl Acad Sci U.S.A. 92(21):9747-51.
22. Hu et al. (2018) “Evolved Cas9 variants with broad PAM compatibility and high DNA specificity”, Nature 556(7699):57-63.
23. Inaba et al. (1992) “Generation of large numbers of dendritic cells from mouse bone marrow cultures supplemented with granulocyte/macrophage colony-stimulating factor”, J Exp Med. 176(6):1693-702.
24. Jinek et al. (2012) “A programmable dual-RNA-guided DNA endonuclease in adaptive bacterial immunity,” Science 337(6096):816-21.
25. Johan et al. (1992) “GLVR1, a receptor for gibbon ape leukemia virus, is homologous to a phosphate permease of Neurospora crassa and is expressed at high levels in the brain and thymus”, J Virol 66(3):1635-40.
26. Judge et al. (2006) “Design of noninflammatory synthetic siRNA mediating potent gene silencing in vivo”, Mol Ther. 13(3):494-505.
27. Kohn et al. (1995) “Engraftment of gene-modified umbilical cord blood cells in neonates with adnosine deaminase deficiency”, Nature Medicine 1:1017-23.
28. Kremer and Perricaudet (1995) “Adenovirus and adeno-associated virus mediated gene transfer”, Br. Med. Bull. 51(1):31-44.
29. Macdiarmid et al. (2009) “Sequential treatment of drug-resistant tumors with targeted minicells containing siRNA or a cytotoxic drug”, Nat Biotechnol. 27(7):643-51.
30. Malech et al. (1997) “Prolonged production of NADPH oxidase-corrected granulocytes after gene therapy of chronic granulomatous disease”, PNAS 94(22):12133-38.
31. Miller et al. (1991) “Construction and properties of retrovirus packaging cells based on gibbon ape leukemia virus”, J Virol. 65(5):2220-24.
32. Miller (1992) “Human gene therapy comes of age”, Nature 357:455-60.
33. Mitani and Caskey (1993) “Delivering therapeutic genes—matching approach and application”, Trends in Biotechnology 11(5):162-66.
34. Nabel and Felgner (1993) “Direct gene transfer for immunotherapy and immunization”, Trends in Biotechnology 11(5):211-15.
35. Nishimasu et al. (2018) “Engineered CRISPR-Cas9 nuclease with expanded targeting space”, Science 361(6408):1259-1262.
36. Remy et al. (1994) “Gene Transfer with a Series of Lipophilic DNA-Binding Molecules”, Bioconjugate Chem. 5(6):647-54.
37. Sentmanat et al. (2018) “A Survey of Validation Strategies for CRISPR-Cas9 Editing”, Scientific Reports 8:888, doi:10.1038/s41598-018-19441-8.
38. Sommerfelt et al. (1990) “Localization of the receptor gene for type D simian retroviruses on human chromosome 19”, J. Virol. 64(12):6214-20.
39. Van Brunt (1988) “Molecular framing: transgenic animals as bioactors” Biotechnology 6:1149-54.
40. Vigne et al. (1995) “Third-generation adenovectors for gene therapy”, Restorative Neurology and Neuroscience 8 (1,2): 35-36.
41. Walton et al. (2020) “Unconstrained genome targeting with near-PAMless engineered CRISPR-Cas9 variants”, Science 368(6488):290-296.
42. Weissman and Kariko (2015) “mRNA: Fulfilling the promise of gene therapy”, Molecular Therapy (9):1416-7.
43. Wilson et al. (1989) “Formation of infectious hybrid virion with gibbon ape leukemia virus and human T-cell leukemia virus retroviral envelope glycoproteins and the gag and pol proteins of Moloney murine leukemia virus”, J. Virol. 63:2374-78.
44. Yu et al. (1994) “Progress towards gene therapy for HIV infection”, Gene Ther. 1(1):13-26.
45. Zetsche et al. (2015) “Cpf1 is a single RNA-guided endonuclease of a class 2 CRIPSR-Cas system” Cell 163(3):759-71.
46. Zuris et al. (2015) “Cationic lipid-mediated delivery of proteins enables efficient protein based genome editing in vitro and in vivo” Nat Biotechnol. 33(1):73-80.

1. A method for modifying in a cell an allele of the Complement component 3 (C3) gene, the method comprising
introducing to the cell a composition comprising:
at least one CRISPR nuclease or a nucleotide sequence encoding a CRISPR nuclease; and
a RNA molecule comprising a guide sequence portion having 17-50 nucleotides or a nucleotide sequence encoding the same,
wherein a complex of the CRISPR nuclease and the RNA molecule affects a double strand break in the allele of the C3 gene.

2. The method of claim 1, wherein the composition further comprises a donor molecule containing a sequence of nucleotides that is introduced at the double strand break site such that the expression of the introduced sequence of nucleotides is mediated by the promoter of the C3 gene.

3. The method of claim 2, wherein the introduced sequence is a sequence from a ATP7B, A1AT, G6PC, SERPINA, LDLR, TTR, ornithine transcarbamylase, argininosuccinic acid synthetase, arginase, argininosuccinase, carbamoyl phosphate synthetase, and N-acetylglutamate synthetase, Alpha Galactosidase A, Coagulation Factor IX, Coagulation Factor VII, Lysosomal Alpha Glucosidase, Fibrinogen, Phenylalanine 4 Hydroxylase, Alkaline Phosphatase, Glucosylceramidase, Beta Galactosidase, Porphobilinogen Deaminase, Arylsulfatase B, Beta Glucuronidase, Alpha N Acetylglucosaminidase, Lysosomal Alpha, Alpha L-Iduronidase, Mannosidase, Phosphatidylcholine Sterol Acyltransferase, N-Sulphoglucosamine Sulphohydrolase, Coagulation Factor X, N-Acetylgalactosamine-6-Sulfatase, Sphingomyelin Phosphodiesterase, A1AT, Iduronate-2-Sulfatase, Lysosomal Alpha Glucosidase, Cyclin Dependent Kinase Like 5, Prolow Density Lipoprotein Receptor Related Protein 1, Phenylalanine Ammonia Lyase, Protein Glutamine Gamma Glutamyltransferase K, or Lysosomal Protective Protein encoding gene.

4. The method of claim 1, wherein the guide sequence portion of the RNA molecule targets the CRISPR nuclease to a SNP position of a C3 allele.

5. The method of claim 4, wherein the SNP position is any one of rs17030, rs199713383, and rs35473940, and/or wherein the SNP position contains a heterozygous SNP.

6. The method of claim 1, wherein the guide sequence portion of the RNA molecule comprises 17-50 contiguous nucleotides containing nucleotides in the sequence set forth in any one of SEQ ID NOs: 1-7,046 that targets a SNP position of a C3 allele.

7. (canceled)

8. The method of claim 1, wherein the RNA molecule comprises a non-discriminatory guide sequence portion that targets Intron 1 of C3 or a 3′ untranslated region (3′ UTR) of C3, and/or wherein the RNA molecule comprises a non-discriminatory guide portion that targets a sequence that is located within a genomic range selected from any one of 19:6677732-6677881 and 19:6719404-6720515.

9. (canceled)

10. The method of claim 1, wherein the guide sequence portion of the RNA molecule comprises 17-50 contiguous nucleotides containing nucleotides in the sequence set forth in any one of SEQ ID NOs: 1-7,046, and/or wherein the guide sequence portion of the RNA molecule comprises 1, 2, 3, 4, or 5 nucleotide mismatches relative to a fully-complementary C3 target sequence.

11. (canceled)

12. The method of claim 1, wherein the guide sequence portion of the RNA molecule comprises 17-50 contiguous nucleotides containing nucleotides in the sequence set forth in any one of SEQ ID NOs: 1-7,046 modified to contain 1, 2, 3, 4, or 5 nucleotide mismatches relative to a fully-complementary C3 target sequence, and/or wherein the guide sequence portion provides higher targeting specificity to the complex of the CRISPR nuclease and the RNA molecule relative to a guide sequence portion that has higher complementarity to an allele of the C3 gene.

13. (canceled)

14. A modified cell obtained by the method of claim 1.

15. The method of claim 14, wherein the modified cell is a stem cell, liver cell, hepatocyte, or iPS-derived hepatocyte.

16. An RNA molecule comprising a guide sequence portion having 17-50 contiguous nucleotides containing nucleotides in the sequence set forth in any one of SEQ ID NOs: 1-7,046, or SEQ ID NOs: 1-7,046 modified to contain 1, 2, 3, 4, or 5 nucleotide mismatches relative to a fully-complementary C3 target sequence.

17. A composition comprising the RNA molecule of claim 16 and at least one CRISPR nuclease.

18. The composition of claim 17, further comprising a donor molecule.

19. The composition of claim 18, wherein the donor molecule contains a sequence from a ATP7B, A1AT, G6PC, SERPINA, LDLR, TTR, ornithine transcarbamylase, argininosuccinic acid synthetase, arginase, argininosuccinase, carbamoyl phosphate synthetase, N-acetylglutamate synthetase, Alpha Galactosidase A, Coagulation Factor IX, Coagulation Factor VII, Lysosomal Alpha Glucosidase, Fibrinogen, Phenylalanine 4 Hydroxylase, Alkaline Phosphatase, Glucosylceramidase, Beta Galactosidase, Porphobilinogen Deaminase, Arylsulfatase B, Beta Glucuronidase, Alpha N Acetylglucosaminidase, Lysosomal Alpha, Alpha L-Iduronidase, Mannosidase, Phosphatidylcholine Sterol Acyltransferase, N-Sulphoglucosamine Sulphohydrolase, Coagulation Factor X, N-Acetylgalactosamine-6-Sulfatase, Sphingomyelin Phosphodiesterase, A1AT, Iduronate-2-Sulfatase, Lysosomal Alpha Glucosidase, Cyclin Dependent Kinase Like 5, Prolow Density Lipoprotein Receptor Related Protein 1, Phenylalanine Ammonia Lyase, Protein Glutamine Gamma Glutamyltransferase K, or Lysosomal Protective Protein encoding gene.

20. A method for modifying a C3 allele in a cell, the method comprising delivering to the cell the composition of claim 18.

21. A method for treating a disorder, the method comprising delivering to a cell of a subject having the disorder the composition of claim 17.

22. (canceled)

23. (canceled)

24. (canceled)

25. The method of claim 21, wherein the disorder is a liver-related disorder or a lysosomal storage disorder.

26. (canceled)

27. A method for treating a disorder, the method comprising delivering to the subject the modified cell of claim 14.

28. The method of claim 27, wherein the disorder is a liver-related disorder or a lysosomal storage disorder.
